(19) United States
(12) Patent Application Publication
(10) Pub. No.: US 2022/0340686 A1
(43) Pub. Date: Oct. 27, 2022

(54) Methods of Assessing Unbound PCSK9 or Effective PCSK9 Activity

(71) Applicant: King's College London, London (GB)

(72) Inventors: Manuel MAYR, London (GB); Sean Anthony BURNAP, London (GB)

(73) Assignee: King's College London, London (GB)

(21) Appl. No.: 17/763,327

(22) PCT Filed: Sep. 25, 2020
(86) PCT No.: PCT/EP2020/076937

§ 371 Date: Mar. 24, 2022

(30) Foreign Application Priority Data
Sep. 25, 2019 (GB) 1913846.0

(51) Int. Cl. C07K 16/40; G01N 33/68; G01N 33/573; G01N 33/92; A61K 45/06; A61K 38/17; A61K 31/7105; A61P 9/10; A61K 39/395; A61P 3/06

(52) U.S. Cl. CPC C07K 16/40; G01N 33/6893; G01N 33/573; G01N 33/92; A61K 45/06; A61K 38/1703; A61K 31/7105; A61P 9/10; A61K 39/3955; A61P 3/06; G01N 2333/96433; C07K 2317/21; C07K 2317/24

(57) ABSTRACT

The invention provides methods of assessing unbound PCSK9 or effective PCSK9 activity in a subject based on the novel insights of the inventors that high levels of PCSK9 are bound to HDL in vivo and that this HDL can activate PCSK9 function. Specifically depleting HDL from a sample from a subject allows improved assessment of the level of unbound PCSK9. The invention provides such analytical methods, plus also associated methods of treatment and related kits.

### TECHNICAL FIELD

The present invention relates generally to methods and materials for use in treating cardiovascular disease by targeting PCSK9.

### BACKGROUND ART

Cardiovascular disease (CVD) remains the leading cause of deaths worldwide, despite major advances in prevention and treatment by lowering low-density lipoprotein cholesterol (LDL-C)(1).

LDL is one class of serum lipoproteins, which comprise a heterogeneous population of lipid-protein complexes. Others include very low (VLDL) and high (HDL) density lipoproteins. Classification is based on differences in particle density related to lipid and protein content. VLDL and LDL are composed of predominately lipid, while high density lipoproteins have a higher content of protein.

Proprotein convertase subtilisin/kexin type 9 (PCSK9), a secreted protein that regulates circulating LDL-C through the hepatic LDL receptor degradation pathway, is a known therapeutic target to further lower LDL-C in patients on maximal statin therapy (18-20).

More specifically, mutations in the PCSK9 gene have been shown to cause hypercholesterolemia, resulting in very high levels of circulating LDL-C and an increased risk of coronary heart disease (CAD) (Abifadel M, Varret M, Rabes J P, Allard D, Ouguerram K, Devillers M, Cruaud C, Benjannet S, Wickham L, Erlich D, Derre A, Villeger L, Famier M, Beucler I, Bruckert E, Chambaz J, Chanu B, Lecerf J M, Luc G, Moulin P, Weissenbach J, Prat A, Krempf M, Junien C, Seidah N G and Boileau C. Mutations in PCSK9 cause autosomal dominant hypercholesterolemia. Nature genetics. 2003; 34:154-6; Fan, D, et al. Self-association of human PCSK9 correlates with its LDLR-degrading activity. Biochemistry 47, 1631-1639 (2008)). Fan et al. used in vitro incubations and transgenic mice models to study this self-association.

The mechanism of action of PCSK9 in the regulation of circulating LDL-C is based on binding to the extracellular domain of the LDL-receptor (LDLR), and upon internalisation of the LDLR bound to an LDL particle, PCSK9 targets the receptor for lysosomal degradation, therefore regulating cell-surface abundance of the LDLR and concomitant circulating LDL levels (35, 18, see also Kwon H J, Lagace T A, McNutt M C, Horton J D and Deisenhofer J. Molecular basis for LDL receptor recognition by PCSK9. Proceedings of the National Academy of Sciences of the United States of America. 2008; 105:1820-5; Cohen J C, Boerwinkle E, Mosley T H, Jr. and Hobbs H H. Sequence variations in PCSK9, low LDL, and protection against coronary heart disease. The New England journal of medicine. 2006; 354:1264-72).

Binding molecules which inhibit the interaction of PCSK9 with LDLR are therefore potential therapeutics.

Circulating PCSK9 is known to exist in a free and bound form in the circulation, and in particular has been shown to bind LDL and lipoprotein(a) (Lp(a)) (21,22). The proportion of bound versus free PCSK9 may be expected to impact on the LDL lowering effects of antibodies to PCSK9.

WO2015/017791 relates to methods for measuring the concentration of “functional” PCSK9 by contacting the sample with a PCSK9-binding agent capable of binding to the LDL-R-binding region of a PCSK9.

WO2018/222186 relates to assays for detecting how much “active” PCSK9 is available in a sample to bind to the LDL receptor, by which is meant PCSK9 that is not already bound to a LDL receptor and is available to bind to a LDL receptor. One aspect of the assays involves the use of LDL receptor and a PCSK9 specific antibody to identify, detect or quantify the PCSK9/LDL receptor complexes.

US2015/0080463 relates to methods in which PCSK9 levels are used to evaluate a patient's expected response to drug treatment for CVD, or to assess risk of CVD and/or cardiovascular events.

US2016/0377618 relates to methods for detecting the level of specific lipoprotein-“bound” level of PCSK9, and optionally “free” PCSK9, lipoprotein or portions thereof and/or PCSK9 unbound lipoproteins present in a biological sample. The patent is particularly concerned with LDL and ApoB.

Given the importance of PCSK9 as a therapeutic target for CVD, it can be seen that the further characterisation of PCSK9's in CVD and its phenotypes, and methods of optimising means of targeting circulating free or functional PCSK9, would provide a contribution to the art.

### SUMMARY OF THE INVENTION

The present inventors have investigated PCSK9-lipoprotein association using various immuno-depletion technologies. They have used nuclear magnetic resonance (NMR)-based lipoprotein profiling, quantitative multiplexed proteomics and targeted lipidomics in the setting of cardiovascular disease and postprandial lipaemia.

Unexpectedly, they have found that the majority of PCSK9 resides on HDL, rather than LDL or Lp(a) as previously believed.

More specifically, correlations of lipoprotein profiles by NMR with plasma PCSK9 levels in a large study revealed an unexpected positive correlation of PCSK9 with medium (8.3-9.3 nm) and especially small-HDL (7.3-8.2 nm) (Otvos J. D. (2002). Measurement of lipoprotein subclass profiles by nuclear magnetic resonance spectroscopy. Clin. Lab. 48 171-180).

Surprisingly, the use of careful ultracentrifugation, column chromatography and immuno-depletion methods targeting apolipoprotein-B and HDL showed that the majority of circulating PCSK9 is not LDL- but HDL-associated. The presence of PCSK9 upon HDL was confirmed in an independent cohort of patients with cardiovascular disease.

Interestingly, different CVD phenotypes also presented different relationships between PCSK9 and HDL. For example, HDL from patients with myocardial infarction (MI) was shown to be enriched in PCSK9 compared to microvascular angina, a change that could not be seen in plasma. Patients with Stable-CAD had higher concentrations of PCSK9 in both HDL and the circulation when compared to patients with MI.

The studies indicate that HDL acts as reservoir of PCSK9 with higher levels in patients on statins and release during postprandial hyperlipidemia.

This new insight that PCSK9 is so abundant on HDL provides the opportunity to more accurately determine “free” PCSK9 which may in turn be used to estimate the PCSK9 available for therapeutic intervention.

Furthermore, it can be seen that HDL-PCSK9 measurement, or measurements taking this relationship into account, provides extra information in relation to CVD status, compared to measuring plasma PCSK9 alone. This information can in turn be used to adapt therapeutic approaches or clinical trials which are based on targeting circulating PCSK9, for example by selection of patient groups or dosage regimens according to the methods described herein.

By way of non-limiting example, and without wishing to be bound by mechanism, patients having a physiological status characterised by relatively higher levels of “free” PCSK9 may be selected as benefiting the most from PCSK9 inhibition, rather than the ones who have relatively higher levels of PCSK9 that is “bound”, since bound PCSK9 may be expected to be less active and/or less available to therapeutics targeting it.

Furthermore, the results herein suggest opposing roles between HDL and LDL in the regulation of LDLR degradation by PCSK9. It is proposed that PCSK9 can bind both HDL (putatively more active PCSK9) and LDL (putatively inhibited PCSK9) within the human circulation and therefore the amount of PCSK9 within each lipoprotein fraction may dictate the overall stimulatory or inhibitory effect upon PCSK9 function, and hence be a useful diagnostic, prognostic or other stratifying measure

The results described herein are particularly unexpected given the focus in the literature on the binding of PCSK9 to LDL.

For example, it has previously been reported that PCSK9 is bound to Lp(a) (Refs 21; 22; also Viney N J, Yeang C, Yang X, Xia S, Witztum J L, Tsimikas S. Relationship between “LDL-C”, estimated true LDL-C, apolipoprotein B-100, and PCSK9 levels following lipoprotein(a) lowering with an antisense oligonucleotide. Journal of clinical lipidology 2018; 12:702-710).

PCSK9 has previously been correlated with HDL levels, albeit inconsistently across studies. For example, PCSK9 levels in patients with CAD reportedly revealed a positive correlation with small HDL-C levels in males but not females (29). Alirocumab treatment for PCSK9 inhibition was shown to shift the HDL-P profiles from small to large sized particles (30). In contrast, genetic inhibition of PCSK9 was associated with a reduction in very large-HDL and a trend towards higher small-HDL particle numbers, highlighting discrepancies between acute inhibition of PCSK9 via antibody treatment and chronic inhibition of PCSK9 by genetics (31).

Kosenko et al (2013) (21) reported in vitro binding studies demonstrated a binding interaction between purified recombinant human PCSK9 and isolated human LDL but not VLDL or HDL.

As noted above, WO02015/017791 relates to methods for measuring the concentration of functional PCSK9 by contacting the sample with a PCSK9-binding agent capable of binding to the LDL-R-binding region of a PCSK9. That disclosure refers to the possibility of removing all (or substantially all) of the LDL from the sample. However, it does not refer to removal of HDL.

As noted above, WO02018/222186 relates to assays for detecting how much active PCSK9 is available in a sample. That disclosure does not refer to binding to HDL, or removal of HDL.

As noted above US2016/0377618 relates to methods for detecting the level of specific lipoprotein-bound level of PCSK9 (free and bound). That disclosure does not refer to binding to HDL, or removal of HDL.

The present invention provides methods of assessing unbound (to HDL) PCSK9 in a subject, for example for use in selecting subjects for treatment, classifying subjects according to their likelihood of responding to treatment, predicting the response of a subject to treatment, determining whether an anti-CVD effect is likely to be produced in a subject by treatment with a compound, and estimating the level of in vivo binding of an antibody directed against PCSK9 in the subject.

The invention further provides methods of personalised or precision medicine where these assessments may be used in clinical trials, or treatments and treatment regimens.

Also provided are kits for use in the methods described herein.

### DETAILED DISCLOSURE OF THE INVENTION

By combining quantitative proteomics and targeted lipidomics in a large collection of HDL samples from patients with CVD, the inventors assessed the impact of PCSK9 on the protein and lipid composition of HDL across multiple CVD phenotypes. They further provided novel insights into HDL remodelling during postprandial hyperlipaemia, with PCSK9 dynamics emerging as a central feature.

Correlations of lipoprotein profiles by NMR with plasma PCSK9 levels in a large study revealed an unexpected positive correlation of PCSK9 with medium and small-HDL.

Thus in one aspect there is provided a method of assessing unbound PCSK9 in a subject the method comprising:

(a) providing a blood sample from the subject who is optionally diagnosed with, or believed to be at risk of, CVD;
(b) specifically depleting at least, or only, HDL from the sample to remove HDL-bound PCSK9 from the sample;
(c) assessing the level of unbound PCSK9 from the depleted sample.

Regarding (c), as explained above, the new insight that PCSK9 is so abundant on HDL provides the opportunity to more accurately determine “free” PCSK9 i.e. that remaining after the HDL depletion.

However optionally step (b) further comprises specifically depleting ApoB and/or LDL from the sample to remove PCSK9 bound to ApoB-containing lipoproteins as well.

As explained below, preferably in step (c) the level of unbound PCSK9 is analysed as a proportion to HDL-bound or total PCSK9.

Optionally in step (b), the method, does not comprise specifically depleting ApoB and/or LDL from the sample to remove PCSK9 bound to ApoB-containing lipoproteins. Optionally only the HDL-bound fraction of PCSK9 is depleted or assessed.

In preferred embodiments the methods may comprise assessing PCSK9 bound to the HDL from the sample. Optionally a ratio of the HDL bound: unbound (or vice versa) or HDL bound: total (or vice versa) or unbound: total may be calculated for use in the methods described herein.

The PCSK9 bound to the HDL from the sample may be compared with the PCSK9 bound to the ApoB and/or LDL in the sample. For example in the methods of the invention a ratio of the two may be derived.

As explained hereinafter, the ratio between the amount of PCSK9 within LDL (putatively inhibited PCSK9) and the amount of PCSK9 within HDL (putatively active PCSK9) may be correlated with an overall measure of PCSK9 activity, and hence provide an additional measure of cardiovascular risk in relation to an individual providing the sample. Therefore, the ratio may be used to stratify patients for treatment e.g. using a PCSK9 inhibitor or other therapy. Similarly, this ratio may be used to could be used to determine the predicted benefit of anti-PCSK9 therapy in a given individual and/or to also assess the response to therapy.

Thus in a further aspect of the invention there is provided a method of assessing PCSK9 activity in a subject, the method comprising:

(a) providing a blood sample from the subject who is optionally diagnosed with, or believed to be at risk of, CVD;
(b) assessing the amount of PCSK9 bound to the HDL from the sample, optionally by specifically depleting HDL from the sample to remove HDL-bound PCSK9 from the sample;
(c) optionally assessing the amount of LDL-bound PCSK9 from the sample, optionally by specifically depleting ApoB and/or LDL from the sample;
(d) correlating the amount of PCSK9 bound to HDL, or the ratio of PCSK9 bound to HDL compared to bound to LDL, with the PCSK9 activity.

By PCSK9 “activity” (or “effective activity” or “function”) is meant the ability of blood PCSK9 to effect reduction of cellular LDLR protein levels and/or propensity of efficiency of PCSK9 uptake and/or multimerisation of PCSK9 on or in cells in the subject, such as hepatocytes. This in turn effects changes in circulating LDL-C levels.

The ratio or relative level may be provided by any of the methods described herein. For example, the PCSK9 bound to the HDL and the PCSK9 bound to the ApoB and/or LDL may be separately isolated and the PCSK9 within each fraction could be measured.

Alternatively, each lipoprotein fraction could be depleted from the sample and in each case the PCSK9 remaining within the depleted sample is measured so as to calculate a percentage of PCSK9 within each lipoprotein fraction.

As explained above the depletion of plasma/serum of all lipoproteins would enable the measure of free-“PCSK9”, which can also be related to PCSK9 activity.

As explained in the Examples below, PCSK9-HDL compartmentalisation was determined during the post-prandial response in healthy volunteers. Postprandial HDL proteome remodelling as assessed by quantitative proteomics revealed a change in the distribution of PCSK9, alongside changes in APOA1 and complement-related proteins.

Immunoassays further confirmed reduction of plasma PCSK9 4 hours after a defined fat meal, coinciding with peak lipaemia, before reverting to baseline levels, a change mirrored in isolated HDL.

These insights raise new implications for the consideration of postprandial responses between individuals when considering PCSK9 as a therapeutic target.

Thus, the methods of the invention PCSK9 in the subject may be assessed postprandially, optionally following a standard meal preceded by a period of fasting. For example, the level of unbound and bound PCSK9 may be assessed over a period of time postprandially, which is optionally up to or equal to 3, 4, 5, 6, 7 or 8 hours.

In one embodiment, to measure the distribution of free vs bound PCSK9 during the postprandial response, participants would be fasted, then given a defined test meal (e.g. containing 50 g fat and 85 g carbohydrate (850 kcal, 15 g protein).

Measurements of free and bound PCSK9 may then be performed before, after and during peak lipaemia, e.g. 4 hours. Thus, permitting the calculation of an area under the curve for PCSK9 release from HDL, that can be used to discriminate between individual postprandial responses.

Subjects

In the methods herein the subject may be individually assessed, for example over a time period.

The subject may be part of a subject group who are optionally diagnosed with, or believed to be at risk of, CVD, all of whom are assessed. This group may be stratified according to the result of the level of unbound PCSK9 from the depleted sample, and optionally the PCSK9 bound to the HDL from the sample. For example, stratified in relation to likelihood of responding to treatment, predicting the response to treatment, determining whether an anti-CVD effect is likely to be produced by treatment with a compound, and estimating the level of in vivo binding of an antibody directed against PCSK9 and so on.

Subjects may be naïve to treatment or may be assessed after a sufficient washout period (e.g., 6-8 weeks without the therapy). The methods may be used to assess the benefit of therapy.

Reference Levels

The methods of the invention may include the step of comparing the assessed level of PCKS9 against a control, reference or threshold level.

The reference level may be based on the PCSK9 bound to the HDL from the sample or total PCSK9 in the sample, wherein optionally the ratio of unbound: HDL bound is calculated.

The threshold level may be a measure of central tendency based on typical levels observed in one or more populations. For example, the threshold level may be a mean level of the functional PCSK9 observed in a given population.

The given population may be defined by one or more of geography, age, ethnicity, sex, and medical history. The threshold level may take into account a measure of variation combined with a measure of central tendency. For example, the threshold level may be a mean level of the functional PCSK9 observed in a given population, plus or minus a margin of error.

A preferred comparator is from a responsive subject, or group of subjects i.e. who have responded positively to treatment with a compound which is a statin or an inhibitor or putative inhibitor of PCSK9 vs. a non-responsive subject, or group of subjects i.e. who have not responded positively to treatment with a compound which is a statin or an inhibitor or putative inhibitor of PCSK9.

Establishing a reference risk score or a “cutoff score for weighted analysis of one or more biomarkers and/or clinical risk factors is known in the art. (Szklo et al., Epide miology: beyond the basics (Second Ed., Sudbury, Mass.: Jones and Bartlett Publishers (2007)); Schlesselman, Case Control Studies (New York: Oxford University Press (1982)); Anderson et al., Cardiovascular disease risk profiles, Am. Heart J., 121:293-8 (1991); Eichler et al., Prediction of first coronary events with the Framingham score: a systematic review. Am. Heart J., 153(5): 722-31, 731.e1-8 (2007); Hoff mann et al., Defining normal distributions of coronary artery calcium in women and men from the Framingham Heart Study, Am. J. Cardiol., 102(9): 1136-41, 1141.el. (2008))

The threshold level may be based on past measurements of functional PCSK9 in the subject. In such embodiments of the method the threshold level could simply be an increase or decrease in functional PCSK9 of a certain amount, or it could be a calculated rate of increase or decrease in functional PCSK9.

The methods described herein may be beneficial in monitoring the progress of therapy in a subject. In this embodiment the method may comprise further step(s) of comparing the PCSK9 levels determined for the sample of interest to one or more PCSK9 levels determined for different samples such as samples taken at different time points for the same subject.

Means of Depleting HDL Bound PCKS9

Means for depleting HDL from a sample are known in the art, for example by using HDL tagging molecules which bind to ApoA1.

Examples of methods may include density-gradient centrifugation, filtration, extraction, immunoprecipitation, etc. provided the method is not such as to displace the bound PCSK9 from the HDL.

Corresponding methods may also be used for depleting ApoB-containing lipoproteins where that is desired. It should be noted that depleting ApoB removes both LDL and Lipoprotein (a) or Lp(a), a subtype of LDL particle.

Preferred methods are as follows:

### 1) Column-based:

Antibody based affinity purification e.g. human HDL-specific IgY affinity columns;
Size exclusion columns, to achieve separation based on size of lipoprotein particles;
High performance liquid chromatography (HPLC) which use a range of different column chemistries, e.g. anion exchange columns.

### 2) Centrifugation:

Ultracentrifugation
Density gradient centrifugation
Inverted rate zonal density gradient (Vertical auto profile)
   3) Electrophoresis, e.g. plasmapheresis
   4) Precipitation methods: e.g. heparin-Mn2+ or dextran sulfate or PEG to deplete apoB containing lipoproteins and use the supernatant for further HDL isolation or measure predominantly HDL-bound PCSK9.

These methods, and their respective advantages and drawbacks, are described in more detail in Hafiane, Anouar, and Jacques Genest. “High density lipoproteins: measurement techniques and potential biomarkers of cardiovascular risk.” BBA clinical 3 (2015): 175-188.

Suitable conditions are also described in the Example Methods hereinafter, which also describe methods of ApoB depletion.

Other methods are provided e.g. in WO2015/175864 which provides methods, kits, and compositions for purifying HDL molecules from a sample (e.g., blood sample) using HDL tagging molecules comprising an HDL lipophilic core binding peptide (e.g., portion of ApoA1) and an affinity tag. The disclosure of that publication, to the extent it concerns means of depleting HDL from samples such as blood or serum, is specifically incorporated herein by reference.

On such other method may comprise:

a) mixing an initial sample (e.g., a sample that is or is not depleted in ApoB/LDL) containing a population of HDL molecules and non-HDL biomolecules with a population of HDL tagging molecules to generate a mixed sample, wherein the HDL molecules each comprise: i) an HDL lipophilic core and ii) a plurality of HDL lipoproteins, and wherein the HDL tagging molecules each comprise: i) an HDL lipophilic core binding peptide, and ii) an affinity tag;
b) incubating the mixed sample such that at least some of the HDL tagging molecules bind to at least some of the HDL molecules thereby generating a population of tagged HDL molecules; and
c) purifying at least a portion of the population of tagged HDL molecules away from the non-HDL biomolecules (and non-tagged HDL molecules) to generate a purified sample, wherein the purifying comprises contacting the mixed sample with a population of capture molecules that are specific for the affinity tag.

Corresponding methods may be used to deplete or measure ApoB/LDL.

Means of Measuring “Free” PCSK9

Means for assessing (measuring, quantifying or assaying) PCSK9 are generally known in the art.

In one embodiment unbound PCSK9 is assessed by assessing PCSK9 bound to the HDL from the sample and subtracting from the total measured in the sample.

A preferred measurement methodology is an enzyme-linked immunosorbent assay (ELISA). ELISA methods are described in the Example Methods hereinafter.

Yet further methods are as follows:

As explained in US2015/0080463, immunoassays can be performed by contacting a sample from a Subject to be tested with an appropriate antibody under conditions such that immunospecific binding can occur if the biomarker is present. Subsequently, detecting and/or measuring the amount of any immunospecific binding by the antibody to the biomarker can then be done. As well as ELISA, other immunoassays include competitive and non-competitive assay systems using techniques such as Western blots, radioimmunoassays, ‘sandwich’ immunoassays, immunoprecipitation assays, immunodiffusion assays, agglutination assays, complement fixation assays, immunoradiometric assays, and fluorescent immunoassays. Both the sandwich immunoassay and tissue immunohistochemical procedures can be highly specific and very sensitive, provided that labels with good limits of detection are used. A detailed review of immunological assay design, theory and protocols can be found in numerous texts in the art, including Butt, Practical Immunology (ed. Marcel Dekker, New York (1984)) and Harlow et al. Antibodies, A Laboratory Approach (ed. Cold Spring Harbor Laboratory (1988)).

CN108424457 describes a monoclonal antibody against PCSK9 and kit containing the same.

WO02015/017791 relates to methods for measuring the concentration of PCSK9 by contacting the sample with a PCSK9-binding agent capable of binding to the LDL-R-binding region of a PCSK9. In the light of the disclosure herein, those methods may be applied to the methods of the present invention, following the step of depleting HDL from the sample. Such methods typically involve

(a) contacting the sample with a PCSK9-binding agent capable of binding to the LDL-R-binding region of a PCSK9 for a period sufficient to allow substantially all of the PCSK9 in the sample to bind to the binding agent; and
(b) measuring directly or indirectly the amount of functional PCSK9 from the sample bound to the binding agent.

As noted above, WO2018/222186 relates to assays for detecting how much active PCSK9 is available in a sample. Such methods typically involve an indirect sandwich ELISA that involves the use of LDL receptor and a PCSK9 specific antibody to identify, detect or quantify the PCSK9/LDL receptor complexes. In one embodiment PCSK9/LDL receptor complexes are formed by adding a sample to a carrier or plate containing the LDL receptor.

As noted above US2016/0377618 relates to methods for detecting the level of specific lipoprotein-bound level of PCSK9 (free and bound). The disclosure discusses the use of gel electrophoresis and immunoassay systems for detecting PCSK9 bound and/or unbound to lipoprotein particles present in a biological sample.

The disclosures of these publications, to the extent they concern means for measuring PCSK9 from bound to lipoproteins, or from depleted samples such as blood, plasma or serum, are specifically incorporated herein by reference.

Other methods for quantification known in the art include using different binders, i.e. aptamers, or binder-independent methods, i.e. mass spectrometry.

Selected Utilities

The methods described hereinabove have utility for the following non-limiting purposes:
selecting a subject for treatment with a compound which is a statin or an inhibitor or putative inhibitor of PCSK9; or
classifying a subject according to their likelihood of responding to treatment with a compound which is a statin or an inhibitor or putative inhibitor of PCSK9; or
predicting the response of a subject to treatment with a compound which is a statin or an inhibitor or putative inhibitor of PCSK9; or
determining whether an anti-CVD effect is likely to be produced in a subject by treatment with a compound which is a statin or an inhibitor of PCSK9; or
estimating the level of in vivo binding of an antibody directed against PCSK9 in the subject.

Other utilities include a method of selecting a dosage regimen for treating a subject diagnosed with, or believed to be at risk of, CVD with a compound which is a statin or an inhibitor or putative inhibitor of PCSK9, wherein the methods of the invention inform the treatment regimen e.g. subjects who may require higher or lower dosages of compound.

The methods may be used to analyse the response of a subject on such therapy. This can help to inform prognosis, treatment duration, or further treatment options.

One particular utility is for assessing the efficacy of a compound which is a statin or an inhibitor or putative inhibitor of PCSK9 which is putatively therapeutic for CVD, the method comprising the steps of:

(a) selecting a treatment group who have been diagnosed with, or believed to be at risk of, CVD and who have been classified as being likely to be responsive to treatment with such a compound according to methods of the invention;
(b) treating members of the treatment group with the compound for a treatment timeframe;
(c) deriving physiological outcome measures for the treatment group;
(d) comparing the outcomes at (d) with a comparator arm of which is optionally a placebo or minimal efficacy comparator arm;
(e) using the comparison in (d) to derive an efficacy measure for the compound.

Treatments of the Invention

The invention also provides novel methods of treatment wherein the assessments described herein are followed by treating a subject selected in accordance with the level of unbound PCSK9 from the depleted sample, and optionally the PCSK9 bound to the HDL from the sample, and optionally the relative amounts of PCSK9 bound to HDL and LDL from the sample, with a compound which is a statin or inhibitor or putative inhibitor of PCSK9.

Thus there is provided:

A method of treating CVD comprising administering a compound which is a statin or an inhibitor of PCSK9 to a subject that has been determined to be responsive to the compound based on the level of serum PCKS9 in the subject not bound to HDL and/or, the relative amounts of serum PCSK9 bound to HDL and LDL.

A method of treating CVD comprising administering a compound which is a statin or an inhibitor of PCSK9 to a subject, wherein the subject has previously been selected for such treatment according to the methods described herein.

A method of treating CVD comprising administering a compound which is a statin or an inhibitor of PCSK9 to a subject, wherein the method comprises selecting the subject for such treatment according to methods described herein.

Also provided are compounds for use in these methods.

Also provided are pharmaceuticals for treatment of CVD in human subjects or patients, the pharmaceutical being a statin or an inhibitor of PCSK9, wherein the patient has been determined to be responsive to the compound based on the level of serum PCKS9 in the subject not bound to HDL.

Also provided are pharmaceuticals for treatment of CVD in human subjects or patients, the pharmaceutical being a statin or an inhibitor of PCSK9, wherein the patient has previously been selected for such treatment according to the methods described herein.

Also provided are pharmaceuticals for treatment of CVD in human subjects or patients, the pharmaceutical being a statin or an inhibitor of PCSK9, wherein the treatment comprises selecting the patient for such treatment according to methods described herein.

Also provided is the use of a compound which is a statin or an inhibitor of PCSK9 in the preparation of a medicament for these treatments.

CVDs

In certain aspects the present invention concerns subjects diagnosed with, or believed to be at risk of, CVD (cardiovascular disease or disorder). Such subjects may therefore be in need to treatment e.g. with a statin or an inhibitor or putative inhibitor of PCSK9.

CVD is used broadly herein to include myocardial infarction (e.g. near-term myocardial infarction), angina pectoris, atherosclerosis, transient ischaemic attacks, stroke, peripheral vascular disease, cardiomyopathy and/or heart failure. It is further intended to include hypercholesterolemia (high levels of total and low-density lipoprotein (LDL) cholesterol), which is a primary cause of atherosclerotic-related diseases.

TABLE T
Anti PCSK9 therapeutics
US2016/0377618 describes PCSK9-targeting therapeutics as follows:

| Name | Company | Therapeutic Type |
|---|---|---|
| Alirocumab | Regeneron/Sanofi | Monoclonal antibody |
| Evolocumab | Amgen | Monoclonal antibody |
| LGT209 | Novartis | Monoclonal antibody |
| RG7652 | Roche/Genentech | Monoclonal antibody |
| Bococizumab | Pfizer | Monoclonal antibody |
| BMS-962476 | Bristol-Myers Squibb | Adnectin |
| ALN-PCS | Alnylam | RNA interference |

Sanrofi/Regeneron's alirocumab and Amgen's evolocumab are described in U.S. Pat. Nos. 8,030,457, 8,563,698, 8,829,165, and 8,859,741.

Other PCSK9-targeting therapeutics include The Medicines Company's Inclisiran (siRNA).

Other PCSK9-neutralizing antibody molecules may also be utilised in the present invention, for example those which bind directly to PCSK9, inhibiting its interaction with LDLR and/or internalisation of LDLR and/or targeting of LDLR for lysosomal degradation.

The term “antibody molecule” when used herein, which term is intended to include any protein having a binding domain which is homologous or largely homologous to an immunoglobulin binding domain. Such proteins may be derived from natural sources, or partly or wholly synthetically produced.

Antibody molecules include antibodies and antibody fragments. For example, an antibody includes monoclonal antibodies, polyclonal antibodies, Fv, Fab, Fab′ and F(ab′)2 immunoglobulin fragments, synthetic stabilized Fv fragments, e.g., single chain Fv fragments (scFv), disulfide stabilized Fv fragments (dsFv), single variable region domains (dAbs) minibodies, combibodies and multivalent antibodies such as diabodies and multi-scFv, single domains from camelids or engineered human equivalents.

An scFv may be comprised within a mini-immunoglobulin or small immunoprotein (SIP), e.g. as described in Li et al. (1997). A SIP may comprise an scFv molecule fused to the CH4 domain of the human IgE secretory isoform IgE-S2 (ε_{S2}-CH4; Batista, F. D., Anand, S., Presani, G., Efremov, D. G. and Burrone, O. R. (1996). The two membrane isoforms of human IgE assemble into functionally distinct B cell antigen receptors. J. Exp. Med. 184:2197-2205) forming a homo-dimeric mini-immunoglobulin antibody molecule.

Antibodies are made either by conventional immunization (e.g., polyclonal sera and hybridomas), or as recombinant fragments, usually expressed in E. coli, after selection from phage display or ribosome display libraries. Methods of providing specific antibodies against different antigens are well established in the art—see e.g. Carvalho, Lucas Silva, et al. “Production Processes for Monoclonal Antibodies.” Fermentation Processes. InTech, 2017.

‘Combibodies’ comprising non-covalent associations of VH and VL domains, can be produced in a matrix format created from combinations of diabody-producing bacterial clones.

Since it is believed that the effectiveness of statins may likewise be affected by the level of “free” PCSK9 in a subject (see e.g. WO2018/222186) it will be understood that all disclosure herein relating to an inhibitor of PCSK9 applies mutatis mutandis to statins.

Suitable statins include, by non-limiting example, atorvastatin, cerivastatin, fluvastatin, lovastatin, mevastatin, pitavastatin, pravastatin, rosuvastatin, and simvastatin. In certain embodiments, the statin is rosuvastatin, which can be administered at a dose between about 5 mg/day and about 40 mg/day. In some embodiments, the statin is atorvastatin, which can be administered at a dose of between about 10 mg/day and about 80 mg/day.

Kits

WO2015/017791 describes apparatus and kits for measuring functional PCSK9, which can be used in conjunction with the features of the invention described herein e.g. for depleting HDL and the instructions for use in accordance with the methods of the invention.

Examples of kit components include:

(a) means for collecting serum from the subject; and/or
(b) means for specifically depleting at least HDL from the sample to remove bound PCSK9 from the sample; and/or
(c) means assessing the level of PCSK9 from the depleted plasma sample; and
(d) instructions for use in the method.
(e) means for specifically depleting ApoB and/or LDL from the sample.

Utilities in Relation to HDL

HDL-C has previously been considered as a therapeutic target per se.

In contrast to LDL-C, levels of high-density lipoprotein cholesterol (HDL-C) are inversely associated to the risk of CVD (2-4). Despite HDL-C being a strong predictor of risk, clinical trials using cholesterol ester transfer protein (CETP) inhibitors to raise HDL-C have failed, with the recent exception of the REVEAL study. The reduction in incidences of major events, however, was probably explained by the additional reduction in LDL-C. Thus, the emphasis on HDL-C as a target for treatment of CVD may have been unwarranted (5-10).

Genome-wide association and Mendelian randomisation studies have cast further doubt on the “HDL hypothesis”, revealing a lack of casual associations between the genetic alteration of HDL-C and CVD outcomes (11). The realisation of the complex interplay that occurs between HDL and other circulating lipoproteins, particularly atherogenic triglyceride-rich lipoproteins such as very low-density lipoproteins (VLDL) has made it challenging to dissect the mechanisms of HDL-mediated protection (12,13).

Besides its well-established role in reverse cholesterol transport, the triglyceride content of HDL is positively associated with CVD risk (14-17). HDL is also protein-rich, with various potential CVD-related functions including apoptosis, inflammation and endothelial dysfunction.

Nevertheless, the novel insights described herein in relation to PCSK9-HDL may further imply new therapeutic opportunities in relation to HDL as well.

### Definitions

As used herein, “sample” refers to a portion of a larger whole to be tested.

As used herein, “blood sample” refers to refers to a whole blood sample or a plasma or serum fraction derived therefrom. In certain embodiment, a blood sample refers to a human blood sample such as whole blood or a plasma or serum fraction derived therefrom.

As used herein, the term “whole blood” refers to a blood sample that has not been fractionated and contains both cellular and fluid components.

As used herein, “plasma” refers to the fluid, non-cellular component of the whole blood. Depending on the separation method used, plasma may be completely free of cellular components, or may contain various amounts of platelets and/or a small amount of other cellular components. Because plasma includes various clotting factors such as fibrinogen, the term “plasma” is distinguished from “serum” as set forth below.

As used herein, the term “serum” refers to whole mammalian serum, such as, for example, whole human serum. Further, as used herein, “serum” refers to blood plasma from which clotting factors (e.g., fibrinogen) have been removed.

Suitably the sample is an in vitro sample.

Suitably the sample is an extracorporeal sample.

In one embodiment suitably the method is an in vitro method or ex vivo method. In one embodiment suitably the method is an extracorporeal method. In one embodiment suitably the actual sampling of the subject (collection of biological sample) is not part of the method of the invention.

Suitably the method does not involve collection of the biological sample. Suitably the sample is a sample previously collected. Suitably the method does not require the presence of the subject whose protein is being assayed. Suitably the sample is an in vitro sample. Suitably the method does not involve the actual medical decision, stricto sensu; such a decision stricto sensu would typically be taken by the physician.

Suitably the method of the invention is conducted in vitro. Suitably the method of the invention is conducted extracorporeally.

Any sub-titles herein are included for convenience only and are not to be construed as limiting the disclosure in any way.

The invention will now be further described with reference to the following non-limiting Figures and Examples. Other embodiments of the invention will occur to those skilled in the art in the light of these.

The disclosure of all references cited herein, inasmuch as it may be used by those skilled in the art to carry out the invention, is hereby specifically incorporated herein by cross-reference.

### FIGURES

FIG. 1. NMR lipoprotein profiling reveals PCSK9-HDL association. NMR lipoprotein analysis was conducted in the community based prospective Bruneck cohort (year 2000 evaluation, n=668). ELISA-based measurements of circulating PCSK9 were correlated against several lipoprotein attributes including; particle number (P), lipid contents (L), phospholipids (PL), total cholesterol (C), cholesterol esters (CE), free cholesterol (FC), triglycerides (TG) and lastly, each lipid class is also represented as a percentage of total lipids (perc).

FIG. 2. Confirmation of PCSK9 association with HDL. ApoB containing lipoproteins were immunoprecipitated from plasma (n=14) and the resulting apolipoprotein profile was quantified by MRM-MS using heavy labelled peptide standards (a), alongside the quantification of PCSK9 by ELISA (b). HDL was immunodepleted from plasma using anti-human HDL specific IgY antibody columns (n=8). The apolipoprotein profile after HDL depletion was quantified by MRM-MS and compared to matched non-depleted plasma (c). The plasma concentration of PCSK9 after HDL depletion was assessed by ELISA (b). 15 ug of non-depleted plasma, HDL-depleted plasma and HDL-fraction were run by SDS-PAGE and total protein was stained to confirm efficient HDL depletion and isolation (n=4 paired representative samples) (e). The presence and enrichment of PCSK9 and APOA1 in the HDL fraction was confirmed by western blot (n=4 paired representative samples) (f).

FIG. 3. The HDL proteome as defined by high resolution mass spectrometry. HDL was isolated from 172 patients with varying CVD-related phenotypes and was analysed by both labelfree and multiplexed TMT proteomic methods. Proteins that were quantifiable in all HDL samples across the CVD cohort by label-free discovery-based MS were considered as the core HDL proteome (n=191, 66 proteins). Proteins are grouped based on Reactome pathway analysis and functionality. Relative contribution to the proteome is estimated through the use of total PSM/mW, taking into account the bias of MS-signal due to protein size. Orange=Apolipoproteins, Pink=Lipid Metabolism, other colours represent related protein clusters. Number represents total Peptide Spectrum Matches/Molecular Weight (PSM/m). Colours are obtained by conditional formatting.

FIG. 4. PCSK9 is a stable member of the HDL proteome. The coefficients of variation in HDL protein abundances, as measured by label-free mass spectrometry, were calculated across the whole cohort (a). Correlations between the MS and ELISA measurements of PCSK9 in HDL (b) and circulating versus HDL-bound PCSK9 levels (c) are represented. Linear regression analysis was used to determine strength of relationship.

FIG. 5. HDL-bound PCSK9 alteration in CVD. ELISA-based quantification of PCSK9 in plasma n=202 and HDL n=167 was conducted and the variation in PCSK9 across the multiple clinical and CVD phenotypes are shown. The variation in plasma and HDL-bound PCSK9 as a result of sex (a, b), statin use (c, d) and as a result of time over a 6-month period post-PCI (e, f) are represented. p-values reported were obtained through the non-parametric Mann-Whitney test, * p<0.05, ** p<0.001. The variation in plasma and HDL-bound PCSK9 across CVD phenotypes is also represented (g, h). The non-parametric Kruskal-Wallis test with Benjamini-Hochberg FDR correction was used; * p<0.05, * p<0.01, ** p<0.001.

FIG. 6. Characterisation of the HDL proteome and lipidome by mass spectrometry.

Proteins correlating with PCSK9 within the core HDL proteome are shown (a), protein correlations are ranked 1 to 65, representing the strength of correlation. The quantitative analysis of the HDL lipidome was conducted using the Biocrates AbsoluteIDQ p400 kits, on a high-resolution Thermo Scientific Q-Exactive HF mass spectrometer. 365 lipid species were quantified in the HDL samples across the cohort (n=149). The sum of each lipid species in a respective class was taken and a Pearson correlation matrix was generated against the HDL apolipoprotein profile, as well as PCSK9 and PLTP. A hierarchical cluster analysis was conducted upon the resulting matrix, being represented in heat map form (b).

FIG. 7. Post-prandial PCSK9 kinetics. Postprandial plasma samples were obtained from 20 individuals at 8, hourly time points. HDL was immuno-precipitated from postprandial plasma samples and label-free quantitative proteomic analysis was conducted on the isolated HDL samples and significant protein changes over the 8 hr time period are represented in a heat map (a). Two distinct clusters of protein changes emerged at at baseline and at 4 hours, and these clusters are graphically represented (b). Significance was determined using the repeated-measure one-way ANOVA test, with Benjamini Hochberg FDR correction.

FIG. 8. PCSK9 is undetectable in human LDL isolated by ultracentrifugation. The PCSK9 content of LDL and HDL from the same individuals with CVD is directly compared (n=16). 10 ug of LDL and HDL diluted 10-fold was used for the ELISA. O.D measurements for PCSK9 in LDL are below the limit of quantification (a). Standard curve O.D values are represented for reference (b).

FIG. 9. Apolipoprotein profile of plasma depleted of lipoproteins. ApoB containing lipoproteins were immunoprecipitated from plasma (n=14) and the resulting apolipoprotein profile was quantified by MRM-MS using heavy labelled peptide standards (a). HDL was immunodepleted from plasma using anti-human HDL specific IgY antibody columns (n=8). The apolipoprotein profile after HDL depletion was quantified by MRM-MS MRM-MS using heavy labelled peptide standards and compared to matched non-depleted plasma (b).

FIG. 10. PCSK9 association with lipoproteins. The level of PCSK9 upon lipoproteins was determined through the use of a modified chemiluminescent ELISA (n=20, 8 time points). PCSK9 was first captured and then antibodies specific for APOB, LP(a) and

APOA1 were used to detect relative levels of PCSK9-lipoprotein association.

FIG. 11. HDL Proteome Correlations. A Pearson correlation matrix was generated for proteins quantifiable in every HDL sample across the cohort (n=191, 66 proteins including PCSK9) and a hierarchical cluster analysis was conducted upon the resulting matrix, being represented in heat map form.

FIG. 21. HDL Proteome Reactome Analysis. The HDL proteome as defined by label-free mass spectrometry was analysed using the open-source Reactome platform (reactome.org). Pathways enriched were ranked based on p-value and the 15 most significant pathways are graphically represented.

FIG. 13. PCSK9 is significantly increased in female HDL, as measured by label-free MS. Females from the CVD cohort used in this study had a significantly increased level of PCSK9 bound to HDL, when measured by label-free MS. Significance was determined by the Mann Whitney U test, with Benjamini Hochberg correction.

FIG. 14. HDL Proteome Correlations. A Pearson correlation matrix was generated for the core HDL proteome as measured by label-free discovery-based mass spectrometry, including only proteins that were quantifiable in every sample across the cohort (n=191, 66 proteins which includes PCSK9). Proteins correlating with APOA1 (a) and Apo(a) (b) are represented, protein correlations are ranked 1 to 65, representing the strength of correlation.

FIG. 15. Characterisation of the HDL lipidome by high resolution mass spectrometry. 365 lipid species were quantified in the plasma and HDL samples of the cohort. The sum of each lipid in a respective class was taken and the percentage contribution to the plasma and HDL lipidome was calculated (a, b).

FIG. 16. Post-prandial PCSK9 kinetics. Postprandial plasma samples were obtained from 20 individuals at 8, hourly time points. The circulating PCSK9 levels were measured by ELISA, alongside clinical measurement of triacylglycerides (TAGs) (a). The relationship between the levels of circulating PCSK9 and TAGs is represented as a linear regression (b). HDL was then immuno-precipitated from postprandial plasma samples and PCSK9 content was measured by ELISA in 8 individuals at 3 time points (c). Significance was determined using the repeated measure one-way ANOVA test, with Benjamini Hochberg FDR correction.

FIG. 17. PCSK9 is enriched in small-HDL. The PCSK9 content of pooled HDL2 (1.063-1.125 g/mL) and HDL3 (1.125-1.210 g/mL) subfractions, obtained from a commercial source, was determined using an anti-PCSK9 ELISA. 10 ug of both HDL2 and HDL3 was used as the input for this assay and each subfraction was run in triplicate.

FIG. 18. PCSK9 is associated with small-HDL and ApoC3. NMR lipoprotein analysis and targeted apolipoprotein profiling was conducted in the Bruneck study (n=656). Plasma PCSK9 levels were correlated with the apolipoprotein profiles as measured by targeted MS with authentic heavy standards.

FIG. 19. HDL-bound PCSK9 modulation during the postprandial response.

(A) Postprandial plasma samples from 20 individuals at 8, hourly time points (validation cohort) were assessed for PCSK9 and HDL-TG content. (B) NMR-based lipoprotein analysis was conducted over the postprandial time course, and the particle concentration of small (S.HDL), medium (M.HDL), large (L.HDL) and extra-large (XL.HDL) HDL are shown. (C) Quantitative label-free proteomics was conducted upon HDL immuno-isolated from postprandial plasma samples (n=8, 3 times points), and significantly changing protein clusters over 8 h are represented graphically. Significance was determined using the non-parametric Friedman test with Dunn's correction, * p<0.05, ** p<0.005, p<0.0005, **** p<0.0001.

FIG. 20. HDL facilities PCSK9 uptake and multimerisation.

(A) PCSK9 and reconstituted HDL (rHDL) were co-incubated prior to HDL immuno-isolation to demonstrate the interaction between rHDL and PCSK9. PCSK9 alone was HDL immuno-isolated as a negative control. LLOD, lower limit of detection. (B) To determine whether HDL can influence PCSK9 cellular uptake, HepG2 cells were treated with His-tagged PCSK9 (5 pg/mL), rHDL or ultracentrifuge-isolated HDL (ucHDL) (25 pg/mL) or a combination of rHDL or ucHDL and HIS-tagged PCSK9 for 6 h prior to immunoblot analysis. (C) Densitometry analysis of three independent replicates using ucHDL are represented. Significance was determined using a t-test with Welch's correction, **** p<0.0001. (D) Recombinant HIS-tagged PCSK9 at a concentration of 1 ug/mL was incubated in the presence of an increasing concentration of rHDL or ucHDL for 24 h at 37° C. The control lane represents PCSK9 incubated without lipoproteins at 4° C. for 24 h. Immunoblot analysis was then conducted, with equal amounts of PCSK9 loading for each sample. Total protein stain is used to visualise ApoA1.

FIG. 21. HDL facilities PCSK9-mediated LDLR degradation.

(A) To determine the ability of HDL to effect PCSK9 action, HepG2 cells were treated with His-tagged PCSK9 (1 pg/mL), ucHDL (50 pg/mL) or a combination of ucHDL and His-tagged PCSK9 for 6 h in the presence of actinomycin D (5 pg/mL), prior to immunoblot analysis. Actinomycin D prevents the compensatory increase in LDLR upon treatment with ucHDL or rHDL without altering the PCSK9 response. (B) Densitometry analysis of three independent replicates are represented, significance was determined using a t-test with Welch's correction, ** p<0.005. (C) The same co-incubation experiment was repeated by isolating the membrane protein fraction through cell-surface biotinylation and NeutrAvidin agarose enrichment, TfR: Transferrin receptor. (D) A working model of the relationship between HDL and PCSK9 is represented. PCSK9 is lost from HDL during postprandial lipaemia, while HDL can positively modulate the uptake and multimerisation of PCSK9, resulting in the enhanced degradation of the LDLR.

### EXAMPLES

### Example 1—NMR Lipoprotein Profiling Identifies PCSK9 Association with HDL

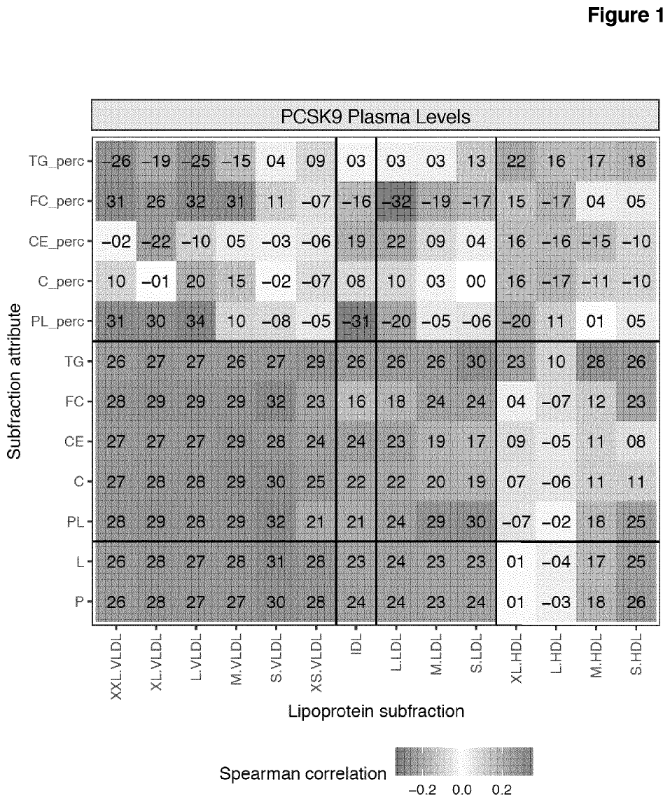

NMR enables the determination of lipoprotein concentrations, alongside their respective lipid content and particle size (17,23,24). We conducted NMR lipoprotein analysis and ELISA measurement of PCSK9 in plasma samples from the community-based prospective Bruneck cohort (year 2000 evaluation, n=668) to determine the relationship between circulating PCSK9 levels and lipoprotein characteristics as measured by NMR. As expected, PCSK9 positively associated with the particle number (P) and lipid content (L) of all circulating VLDL, IDL and LDL particles (FIG. 1). However, PCSK9 revealed a surprisingly strong positive association with the particle number of S-HDL (Pearson correlation=0.26, p<0.001).

### Example 2—PCSK9 is Predominantly Associated with HDL

Previous reports have suggested that PCSK9 binds to LDL (21) and Lp(a) (22).

To determine whether PCSK9 is predominantly associated with APOB-containing lipoproteins or HDL, we performed immunodepletion experiments for APOB and APOA1 from human plasma (n=14)

Immunodepletion of APOB resulted in a 99% reduction in APOB (FIG. 29). As expected, a similar depletion efficiency was observed for Lp(a), an LDL particle that carries apolipoprotein(a) as an additional protein component.

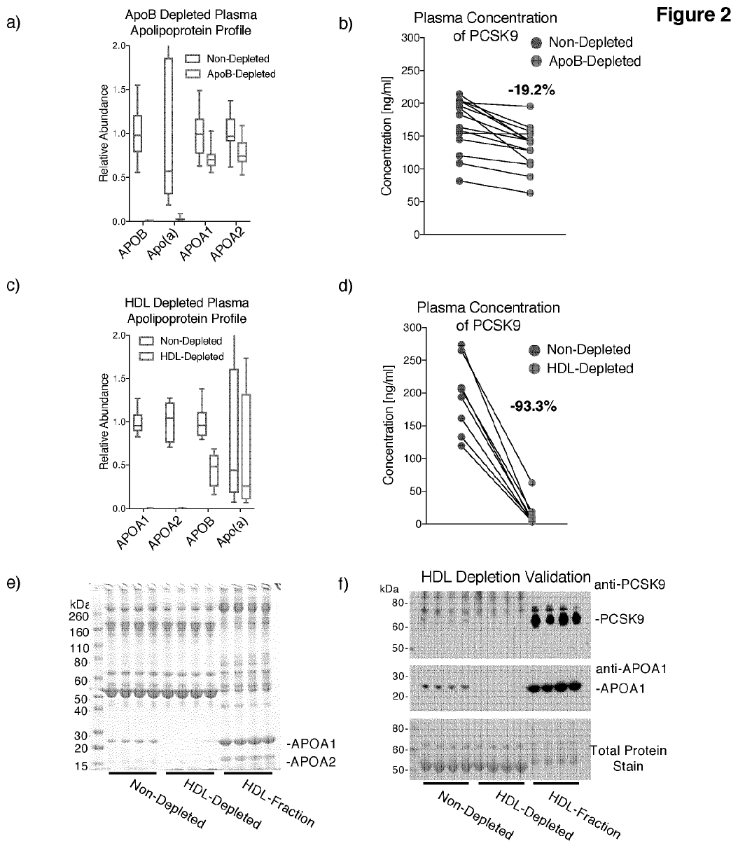

After APOB depletion, the plasma concentration of PCSK9 was reduced by less than 20%, suggesting that contrary to current assumption most circulating PCSK9 is not LDL or Lp(a)-associated (FIG. 2b). In fact, PCSK9 was below the limit of quantification by ELISA in isolated LDL from human plasma (n=16) (FIG. 8).

Next, APOA1 was immuno-depleted (n=8), again resulting in a 99% reduction of APOA1, confirming a highly efficient depletion (FIG. 2c). APOA2, another abundant HDL protein, showed a similar reduction. Notably, PCSK9 plasma levels revealed an over 90% reduction upon HDL depletion (FIG. 2d).

Full plasma apolipoprotein profiles post-lipoprotein depletions are shown in FIG. 9. Efficient HDL depletion and PCSK9 enrichment in the HDL-fraction were further confirmed by immunoblotting (FIG. 2e, ). Furthermore, the determination of PCSK9 association with lipoproteins through an independent antibody-based apolipoprotein capture method revealed a much greater abundance of PCSK9 associated with APOA1, when compared to APOB and apolipoprotein(a) (FIG. 10). Thus, PCSK9 is predominantly associated with HDL rather than LDL or Lp(a).

### Example 3—the HDL Proteome in Patients with CVD

Further interrogation of the HDL proteome was conducted in 172 patients with different CVD (Table 1), namely:
microvascular angina,
stable coronary artery disease (CAD),
myocardial infarction (MI) and
stable CAD with percutaneous coronary intervention (PCI)

TABLE 1
Cohort Patient Characteristics for HDL isolation

| | Microvascular | | | PCI with | p- |
|---|---|---|---|---|---|
| | Angina | Stable-CAD | MI | follow up | value |
| n (% Total) | 18 (10.5) | 66 (38.4) | 56 (32.6) | 32 (18.6) | |
| Age(±SD) | 57.6 ± 10.93 | 64.43 ± 14.65 | 65.45 ± 9.05 | 62.76 ± 9.23 | 0.05 |
| Males (%) | 6 (33.3) | 43 (65.2) | 31 (55.4) | 24 (75.0) | 0.06 |
| Current Smoker (%) | 1 (5.9) | 9 (13.6) | 16 (30.8) | 4 (12.5) | 0.07 |
| History of Diabetes(%) | 2 (12.5) | 17 (25.8) | 16 (31.3) | 5 (15.6) | 0.45 |
| Statin Use | 11 (61.1) | 62 (93.9) | 13 (30.2) | 29 (93.5) | <0.001 |

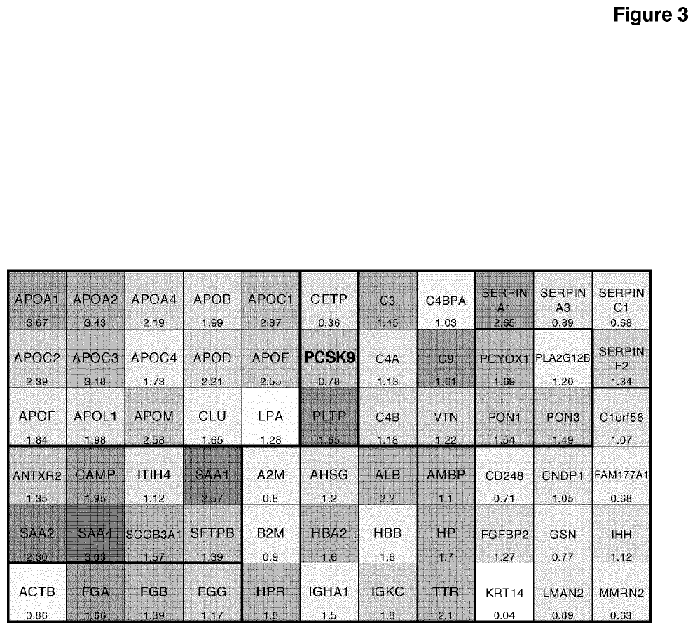

For the last subgroup, a 6-month follow-up post-PCI was included (n=32). Initially, discovery-based mass spectrometry (MS) was used to obtain a comprehensive overview of the proteome. To determine inter-protein relationships only proteins quantifiable in all HDL samples were retained. This core HDL proteome of 66 proteins includes PCSK9 and is represented in table form based on functionality (n=191, FIG. 3). A Pearson correlation matrix was generated for these 66 proteins, revealing the distinct clusters of HDL-associated proteins (FIG. 11).

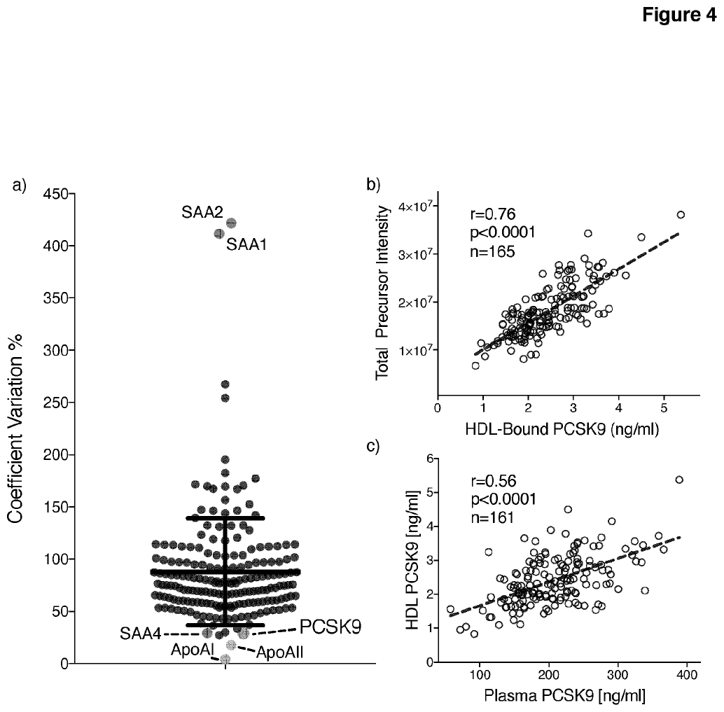

Similar to APOAI, PCSK9 showed remarkable stability in abundance across HDL samples (FIG. 4a). In contrast, key drivers of the variation in the HDL proteome were inflammatory-related proteins, such as acute-phase proteins serum amyloid A1 and serum amyloid A2. The main pathways returned by Reactome analysis on the HDL proteome in CVD patients were related to lipoproteins, interferon signalling, platelet and neutrophil degranulation, fibrin clotting and complement activation (FIG. 12).

To validate the quantitative accuracy of the MS measurement, PCSK9 was measured by ELISA. MS and ELISA-based quantification of PCSK9 in HDL were highly correlated (r=0.76, n=165, FIG. 4b). A weaker correlation was observed when comparing circulating versus HDL-bound levels of PCSK9 (r=0.56, n=161, FIG. 4c). Thus, HDL-bound PCSK9 might provide additional information.

### Example 4—HDL-Bound PCSK9 in Patients with CVD

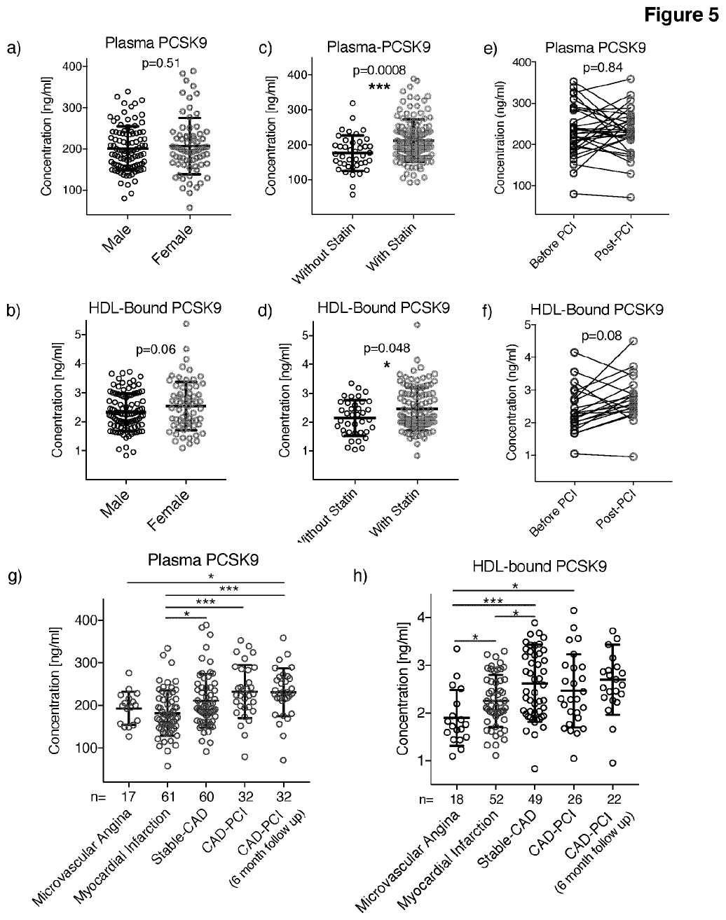

Circulating PCSK9 levels are influenced by sex and statin use (25,26). In our cohort of patients with CVD, plasma PCSK9 levels were similar between males and females (FIG. 5a). HDL-bound PCSK9, however, was significantly increased in females compared to males in the label-free MS analysis (FIG. 13), and a similar trend was observed using PCSK9 immunoassays (p=0.06) (FIG. 5b). In contrast, a highly significant increase in plasma PCSK9 levels in patients taking statins (p=0.0008) was mirrored in the HDL fraction but only with borderline significance (p=0.048) (FIG. 5c, d). Among the various CVD manifestations, patients with stable CAD had the highest concentrations of PCSK9. In patients with 6-month follow-up post-PCI (n=32), PCSK9 levels in both plasma and HDL were stable over time (FIGS. 5e, f). Patients with microvascular angina had lower levels of PCSK9 on HDL compared to patients with MI and stable CAD, a difference that was not evident in plasma (FIG. 5g, h).

### Example 5—PCSK9 on HDL Correlates with PLTP and Complement Factors

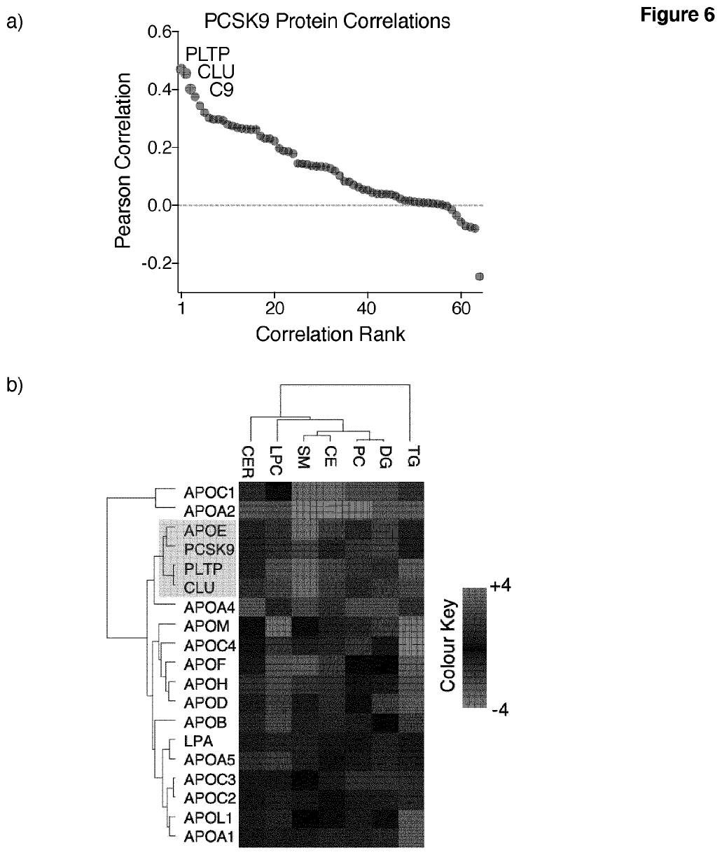

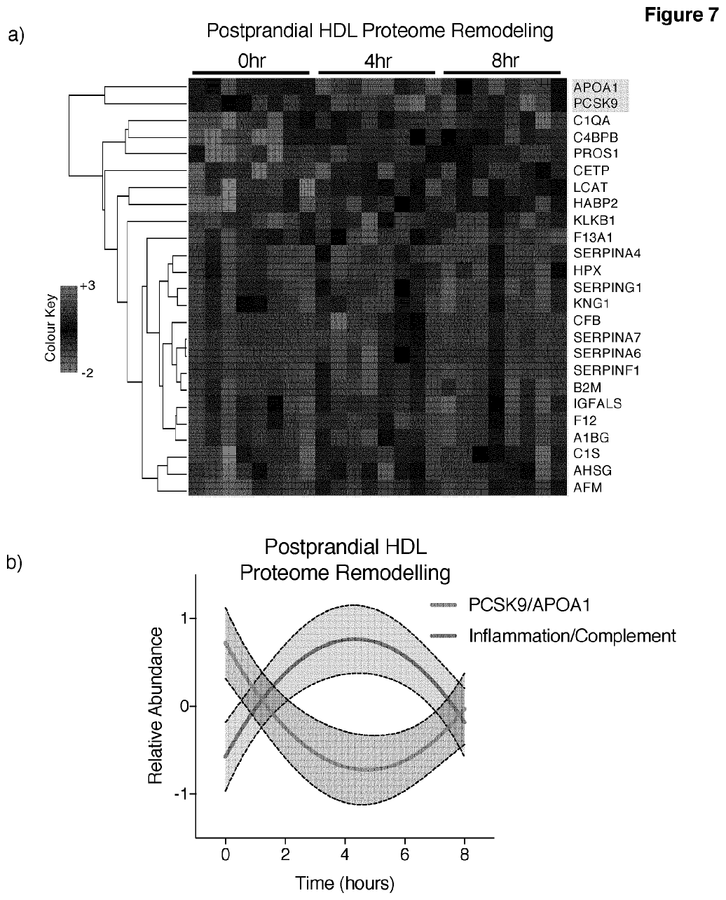

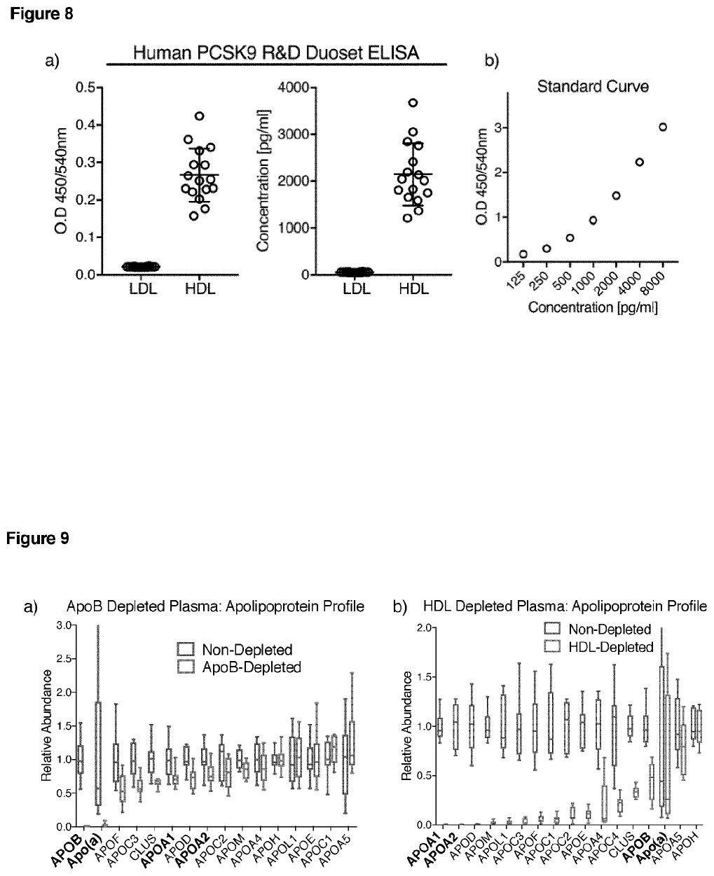

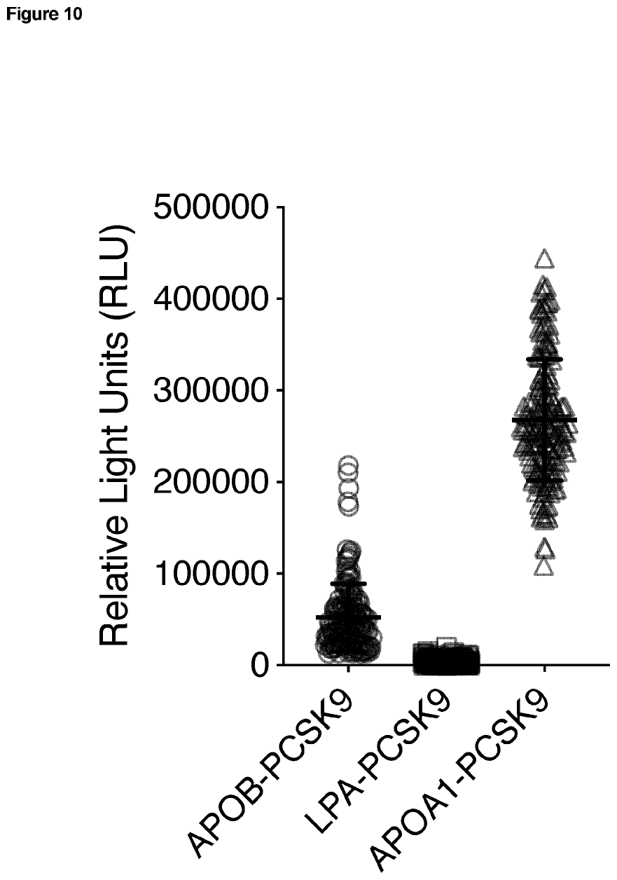

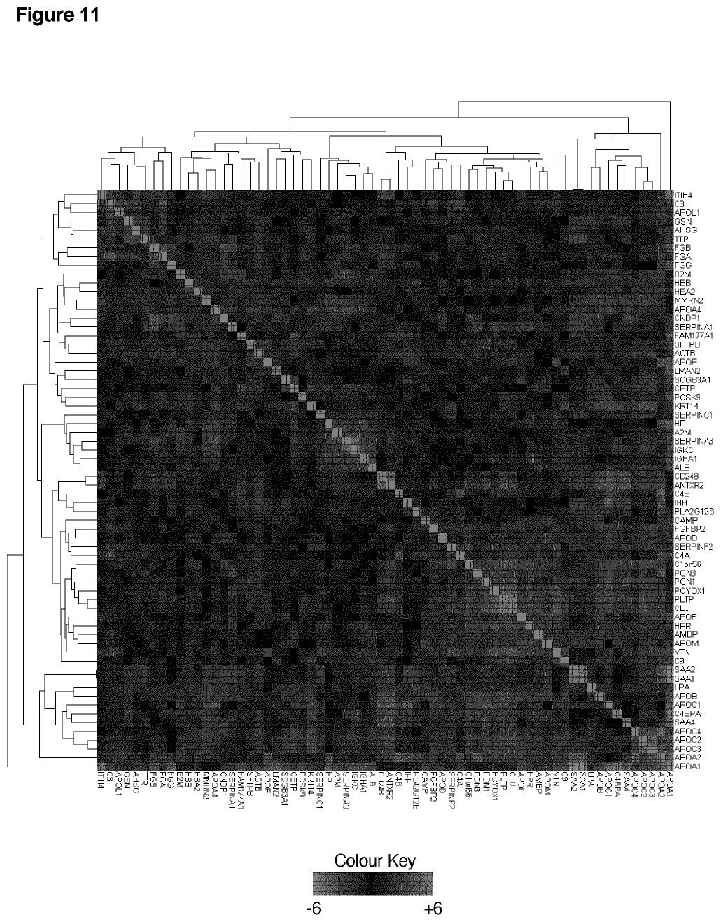

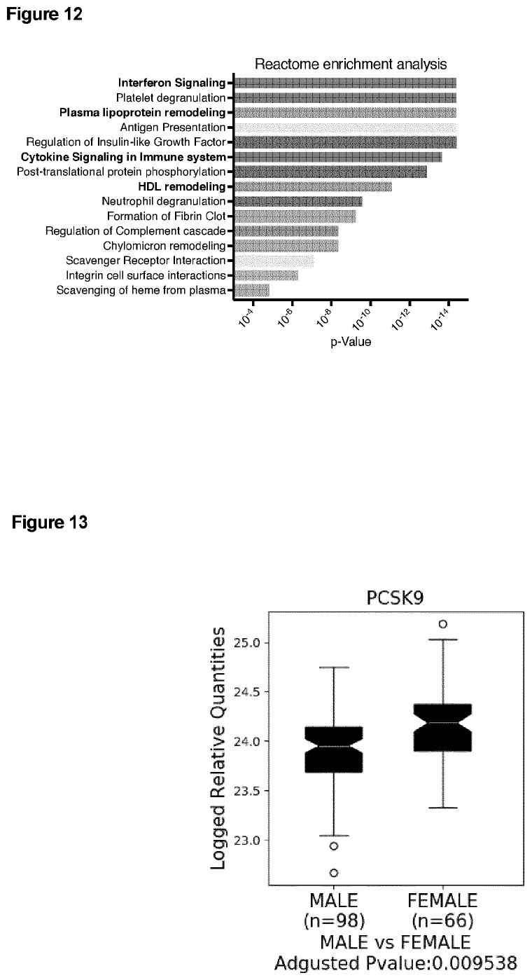

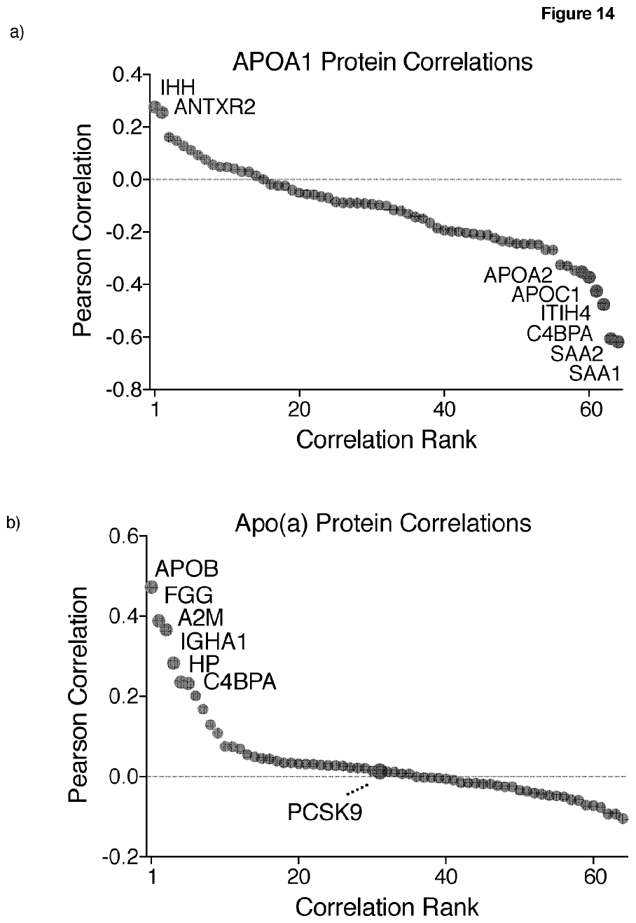

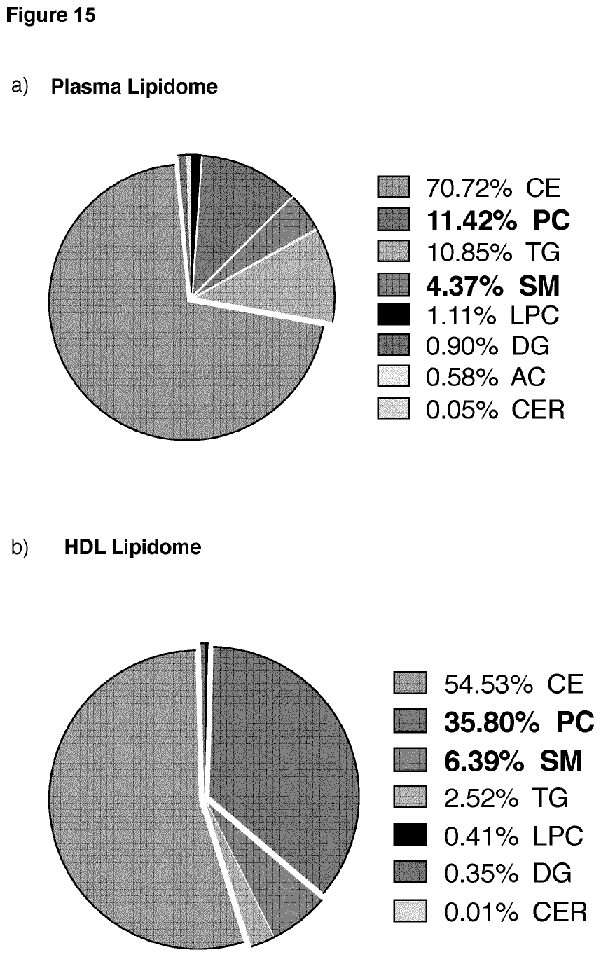

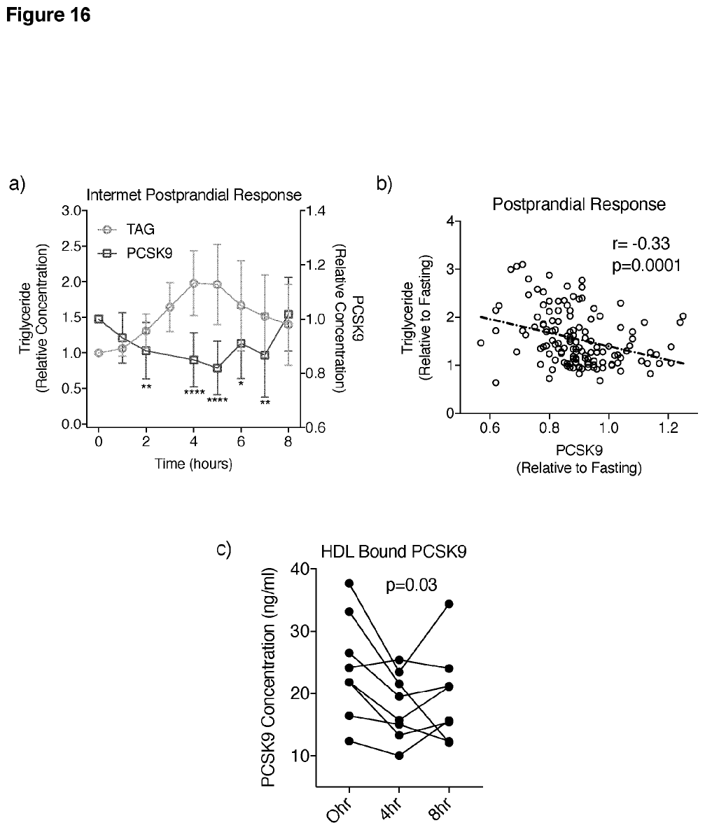

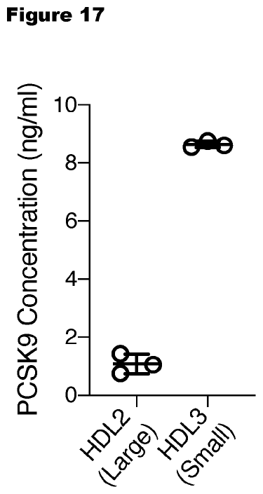

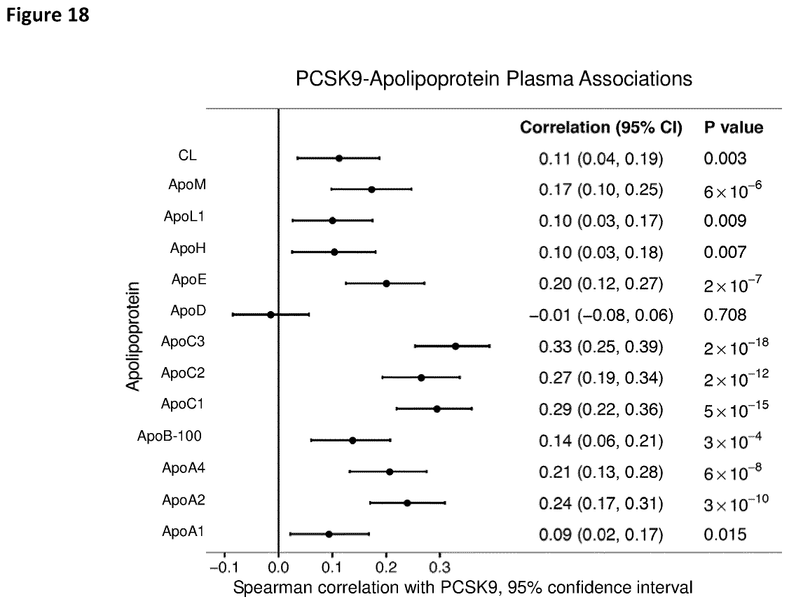

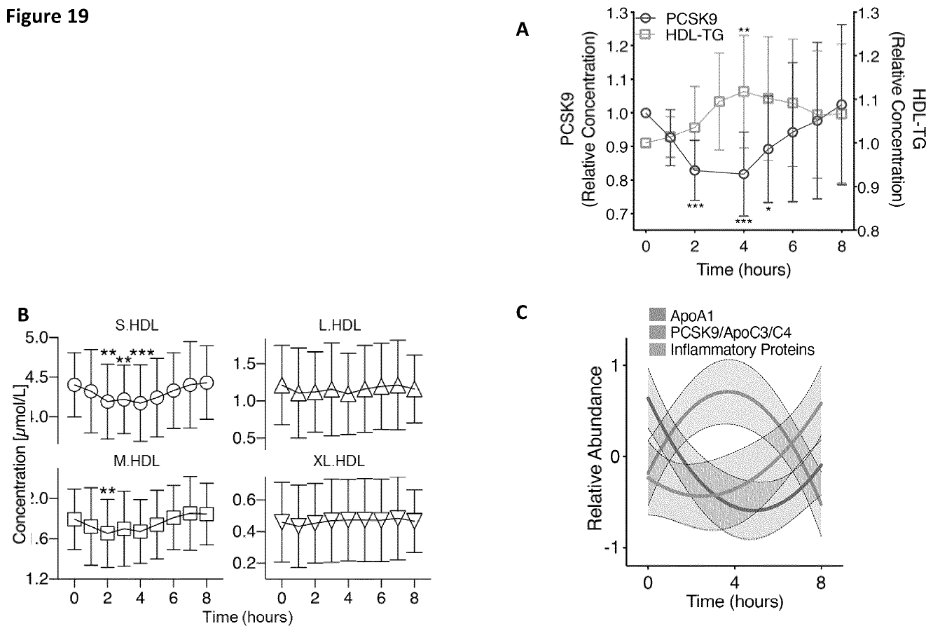

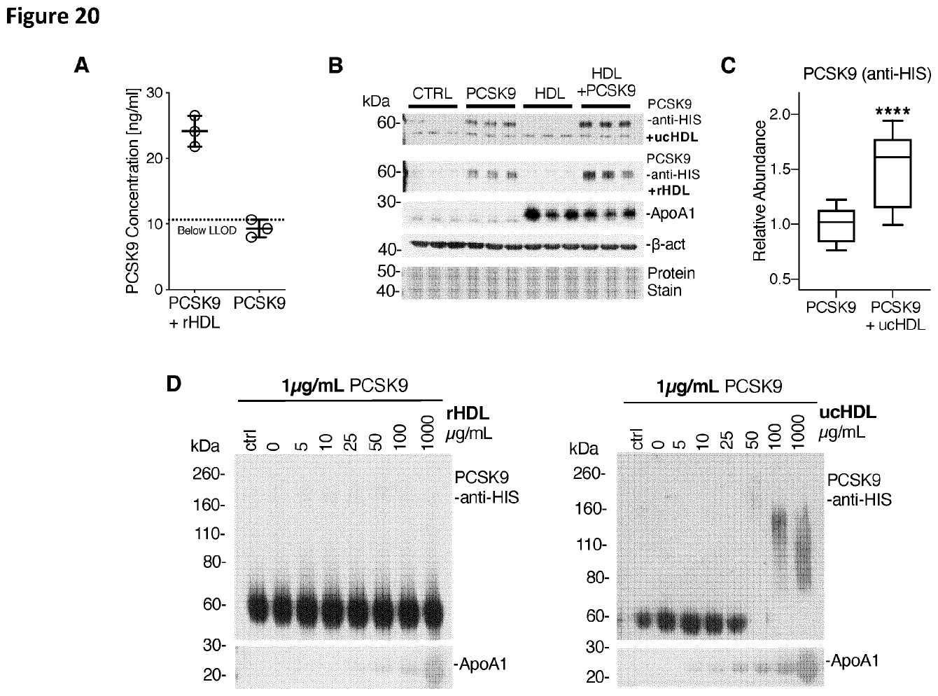

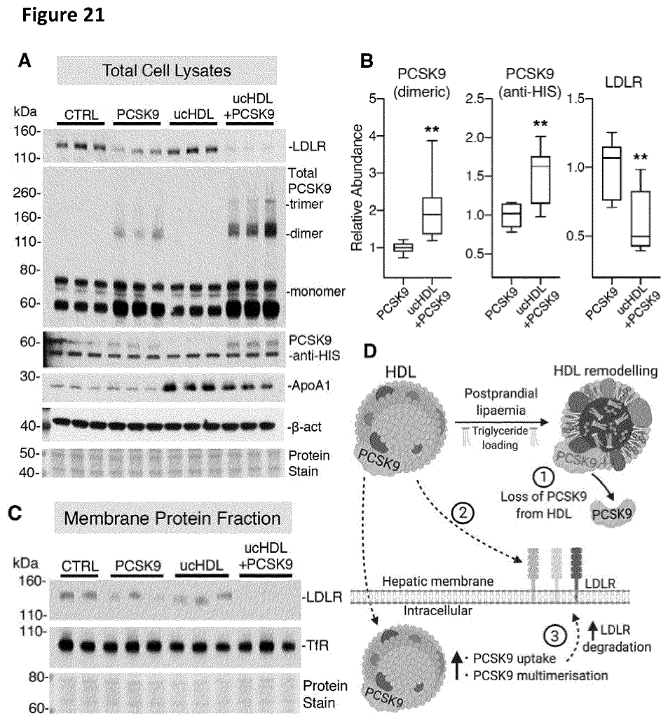

PCSK9 on HDL showed a strong positive association with phospholipid transfer protein (PLTP), the key protein responsible for exchanging lipids between VLDL and LDL to mature HDL (27). Other proteins that were positively correlated with PSCK9 were proteins involved in complement formation (Clusterin—CLU, complement factor 9—C9) (FIG. 6a). Lastly, strong clustering between APOB and LPA revealed the minor presence of Lp(a), a lipoprotein particle known to overlap in density with HDL(28). Proteins that strongly correlated with apolipoprotein(a) revealed an Lp(a) protein signature (FIG. 11, 14b). There was no correlation between apolipoprotein(a) and PCSK9 in the HDL proteome (FIG. 14b).

### Example 6—Comparison to the HDL Lipidome in Patients with CVD

The strong positive association between PCSK9 and lipid metabolism related proteins prompted us to interrogate the associations of lipid species with PCSK9. Targeted quantitation of 365 lipid species was conducted in HDL from the entire cohort. Cholesterol esters (CE) and phosphatidylcholine (PC) were the most abundant lipid species in HDL, contributing approximately 90% of total lipid content (FIG. 15a, b). Another major constituent of the HDL lipidome were sphingomyelins (SM) (FIG. 15b).

Hierarchical cluster analysis on correlation matrices between the apolipoprotein profile and lipidome in HDL replicated the proteome-based clustering of PCSK9 with PLTP and clusterin but included apolipoprotein E (APOE) as well. When correlated with the HDL lipidome, this protein cluster revealed a strong positive association with SM (Pearson correlation=0.4, P<0.0001) (FIG. 6b).

### Example 7—the Effect of Food Intake on Postprandial HDL Remodelling

Lastly, postprandial HDL remodelling was evaluated by proteomics. The test meal contained 50 g fat and 85 g carbohydrate (850 kcal, 15 g protein). A 50 g fat load has been shown to be the optimum quantity to discriminate between individual postprandial responses. A large cluster of inflammatory proteins increased in abundance upon HDL at the 4 hr time point before normalising to fasted levels (FIG. 7a, b, Cluster 2). In contrast, a postprandial reduction in HDL-bound PCSK9 was revealed by proteomics, alongside a reduction in APOA1 (FIG. 7a, b, Cluster 1).

PCSK9 measurements by immunoassays confirmed a reduction of circulating PCSK9 levels compared to the fasted state. This reduction coincided with peak postprandial lipaemia, within the first 5 hrs. Circulating PCSK9 and triglyceride levels reverted back to baseline concentrations at 8 hrs post-prandially (FIG. 16a, n=20, 8 time points). A highly significant, negative correlation was observed between the postprandial PCSK9 and triglyceride responses (r=−0.33, p=0.0001, FIG. 16b). The postprandial change in HDL-bound PCSK9 mirrored the changes observed in the circulation (FIG. 16c, n=8, 3 time points). These findings uncovered dynamic changes in PCSK9 during the postprandial remodelling of the HDL protein content.

The plasma reduction of PCSK9 was replicated in a second postprandial cohort, adhering to the same test meal (8 time points, n=20, FIG. 19A). Additionally, NMR-based lipoprotein analyses were conducted in the postprandial validation cohort and revealed triglyceride loading of HDL (FIG. 19A) as well as a significant reduction in the particle concentrations of S.HDL and M.HDL between 2-4 hours when lower PCSK9 levels were observed (FIG. 40). In contrast, L.HDL and XL.HDL remained unchanged (FIG. 190). Next, HDL was immuno-isolated over the postprandial time course at 0 h, 4 h and 8 h (n=8 each) and analysed by quantitative proteomics. Consistent with previous results, ApoA1 content of HDL was greatest at 8 h postprandially (FIG. 4C).¹⁵ In contrast, a cluster of inflammatory proteins increased in abundance upon HDL at 4 h postprandially, before returning to fasted levels. The postprandial reduction in HDL-bound PCSK9 as confirmed by proteomics was accompanied by a reduction of C apolipoproteins, including ApoC3.

### Example 8—HDL Facilitates PCSK9-Mediated LDLR Degradation

Lastly, we interrogated whether HDL can alter PCSK9 function. Recombinant PCSK9 was capable of associating with rHDL in vitro (FIG. 20A). Treatment of cells with recombinant PCSK9 and either rHDL or ucHDL increased PCSK9 uptake compared to treatment with PCSK9 alone (FIG. 20B, C). In contrast, ucHDL but not rHDL was able to promote multimerisation of PCSK9 (1 μg/mL) at physiological concentrations (FIG. 50). Treatment of HepG2 cells with PCSK9 and ucHDL reduced cellular LDLR protein levels to a greater extent as compared to PCSK9 alone (FIG. 21A, B). This coincided with higher uptake and increased multimerisation of PCSK9 (FIG. 21A, B). Both total and cell membrane LDLR levels were reduced upon incubation of cells with PCSK9 and ucHDL (FIG. 21C).

The functional relationship between HDL and PCSK9 is schematically represented in FIG. 21D.

### Example 9—Discussion of Examples 1-8

The data presented in this study provide the first proteomics evidence that PCSK9 is found upon HDL. Combining findings from a prospective, community-based study with findings in isolated HDL from CVD patients and in healthy volunteers during the postprandial phase, it demonstrates that the majority of circulating PCSK9 is associated with HDL, overturning the prevailing assumption that PCSK9 is bound to APOB-carrying lipoprotein particles such as LDL and Lp(a). Instead, HDL acts as reservoir of PCSK9 with higher levels in patients on statins and release during postprandial hyperlipidemia.

Plasma PCSK9 levels correlate to small HDL. The combination of NMR lipoprotein profiling alongside quantitative PCSK9 measurement in a prospective community-based cohort revealed the relationships between PCSK9 and lipoprotein subpopulations. The positive correlation of PCSK9 with the particle number and lipid content of small-HDL (S-HDL-P/L) was similar to the strength of correlation seen with VLDL and LDL. The correlation analyses also revealed a strong positive association between PCSK9 and the triglyceride content of HDL, a component of HDL recently associated with CVD-risk (17).

For validation, measurements of PCSK9 were performed in isolated small, dense HDL (HDL3) and larger, less dense HDL (HDL2), confirming an enrichment of PCSK9 within HDL3 (FIG. 21).

Next, PCSK9 measurements were correlated with plasma apolipoprotein measurements by targeted mass spectrometry (MS) (FIG. 18)¹³. PCSK9 plasma levels showed a surprisingly strong correlation with C apolipoproteins, in particular with ApoC3, an inhibitor of lipoprotein lipase, the enzyme primarily responsible for the hydrolysis of plasma triglycerides (FIG. 18)²⁴.

PCSK9 is actually associated with HDL. Previous publications suggested that PCSK9 was circulating partly in association with LDL and Lp(a), with reports suggesting up to 40% of PCSK9 to be LDL-associated (21,22). However, in our study circulating PCSK9 levels were only reduced by <20% upon APOB and Lp(a) removal. Through the use of immuno-depletion technologies for APOA1, the major apolipoprotein of HDL, we demonstrated a striking reduction of plasma PCSK9 levels, suggesting a predominant interaction of PCSK9 with HDL rather than LDL and Lp(a). The presence of PCSK9 upon HDL was further confirmed in isolated HDL in two separate large-scale human cohorts, in which HDL was isolated either through ultracentrifugation or immuno-isolation and consistently detected by MS. In contrast, Romagnuolo et al were unable to demonstrate in vitro binding between isolated Lp(a) and PCSK9, an association that has only been observed in patients with extremely high levels of Lp(a) (22,32). Furthermore, the in vitro study first identifying PCSK9 to associate with LDL also could not detect PCSK9 in LDL isolated from normolipidemic human plasma, an observation deemed due to salt concentration and high centrifugal force causing a loss of PCSK9 from LDL during isolation (21). Our data concur with the latter interpretation as the measured content of PCSK9 upon HDL isolated through ultracentrifugation compared to that measured in immuno-isolated HDL was almost 10-fold lower (33). Lastly, human lipoprotein apheresis studies determined a 50% reduction in PCSK9 upon the removal of 77% and 89% of LDL and Lp(a) respectively, however a significant 18% reduction in HDL was also present (34,35). Our data would suggest that HDL removed during apheresis was a contributor to the PCSK9 reduction observed.

HDL as an endogenous reservoir of PCSK9. The core function of PCSK9, and the rationale for therapeutic targeting, is its downregulation of hepatic LDLR surface expression, thereby raising circulating levels of atherogenic VLDL, IDL and LDL particles (18,20,36,37). PCSK9 not only regulates the LDLR through binding to the extracellular region of this receptor but can also control LDLR degradation within the cell (38). PCSK9 has also been shown to regulate the production of triglyceride-rich lipoproteins in both the liver and intestine, through an LDLR-dependent and independent manner (39-42).

Without wishing to be bound by theory, and in light of the findings herein it could be envisaged that HDL acts as a PCSK9 reservoir in the circulation and that internalisation of HDL could deliver a pool of PCSK9 to the intracellular environment, that can then influence the pathways outlined above. Alternatively, differential PCSK9 compartmentalisation could modulate its activity.

Furthermore, unlike LDL that is thought to inhibit PCSK9 function upon the LDLR²¹, our data suggest, that at physiological concentrations of PCSK9 and HDL, HDL promotes the multimerisation of PCSK9 in a dose-dependent manner. The multimeric state of PCSK9 has previously been associated with its LDLR degrading capabilities, therefore a varying ratio of LDL and HDL within the human circulation could determine the activity of PCSK9 (Fan et al. supra).

Postprandial changes in PCSK9 compartmentalisation. Of the limited human postprandial studies to date that investigate the PCSK9 response, our study is the first to reveal PCSK9 to be significantly reduced in the circulation postprandially, with previous reports highlighting a trend of reduction (43,44). The change in PCSK9 was mirrored when analysing its abundance in the HDL fraction. Intriguingly, although no change in APOA1 was detected when analysing the plasma over this postprandial time course, a reduction was observed in the APOA1 content of HDL, a change mimicking that of PCSK9. The change in APOA1 content of HDL, independent of total HDL variation, would suggest a subpopulation remodelling of HDL over this postprandial period. Previously, NMR-based lipoprotein analysis of plasma over the postprandial response in humans revealed a reduction in small-HDL, versus a concomitant increase in medium-HDL particle number. It was also revealed in this study that women had a greater shift in HDL subpopulation redistribution when compared to men, a sex difference that may be apparent in our postprandial study, particularly due to the known effects of gender on circulating PCSK9 (25,45,46).

Associations of lipid and inflammatory HDL proteins with PCSK9. PCSK9 abundance strongly correlated with lipid and complement-related proteins, including PLTP and CLU respectively. PLTP has been shown to be able to directly bind PCSK9, in vitro, emphasising the validity of the positive correlation seen between the two proteins within the HDL proteome in this study (47). Besides PLTP and CLU, PCSK9 was also associated with APOE in respect to the HDL lipidome pointing towards an involvement of PCSK9 in the lipid modelling of HDL.

PCSK9 also strongly correlated with known regulators of the complement cascade within the HDL proteome (48). CLU inhibits the formation of the membrane attack complex through the interruption of C9/C5b-C8 and C5b-C7 complex formation, respectively (49). PCSK9 positively associated with C9 within the HDL proteome, further suggesting its role in the regulation of the complement cascade. The possible interaction between PCSK9 and PLTP upon HDL is intriguing in this respect due to the arising role of PCSK9 in the immune response, particularly in the clearance of pathogen (50,51). Postprandial remodelling of the HDL proteome also saw a large cluster of protein changes related to the complement cascade, highlighting an inflammatory process implicated in postprandial lipaemia that could involve PCSK9.

Conclusions. This study for the first time provides proteomic evidence that HDL is the main carrier of PCSK9 in the circulation, and that this association is dynamic during the human postprandial response.

In the light of the results presented herein it is plausible that PCSK9 released from HDL, a facilitator of PCSK9-mediated LDLR degradation, may bind ApoB-containing lipoproteins that are known to inhibit PCSK9 function, and therefore control the hepatic uptake of triglycerides in the postprandial phase.

Our study for the first time reveals that HDL is capable of modulating the LDLR-degrading capacity of PCSK9 in vitro. In an HepG2 cell system the addition of PCSK9 with HDL (isolated by ultracentrifugation) led to a greater reduction in LDLR protein levels when compared to cells treated with PCSK9 alone, in both whole cell lysates and membrane fractions (FIG. 21). This positive regulation of PCSK9 function could be attributed to the observed promotion of uptake and multimerisation of PCSK9 when HDL was present, when compared to cell treatment with PCSK9 alone (FIG. 21B).

It has previously been shown that LDL is capable of negatively regulating the LDLR-degrading capacity of PCSK9. PCSK9-driven LDLR degradation was shown to be dose-dependently inhibited by LDL. These authors reveal also that the presence of LDL with PCSK9 reduced PCSK9 uptake by the cells, in comparison to cells treated with PCSK9 alone (FIGS. 6 and 7 within the paper cited)²¹.

In the light of the results herein, it is plausible that that there are opposing roles between HDL and LDL in the regulation of LDLR degradation by PCSK9. PCSK9 can bind both HDL and LDL within the human circulation and therefore the amount of PCSK9 within each lipoprotein fraction could dictate the overall stimulatory or inhibitory effect upon PCSK9 function.

It follows that the ratio between the amount of PCSK9 within LDL (inhibited PCSK9) and the amount of PCSK9 within HDL (active PCSK9) may be used as an overall measure of PCSK9 activity and therefore may provide an additional measure of cardiovascular risk in a given individual, particularly given the fact that total levels of PCSK9 have proved inconclusive as an independent predictor of atherosclerotic risk⁵². Furthermore, the use of this ratio measure of PCSK9 activity could be used to determine the predicted benefit of anti-PCSK9 therapy in a given individual and to also assess the response to therapy.

### Methods for Examples 1-9

Bruneck Cohort

The Bruneck Study is a community-based, prospective survey of the epidemiology and pathogenesis of atherosclerosis and cardiovascular disease (1,2). At the 1990 baseline evaluation, the study population comprised an age- and sex-stratified random sample of all inhabitants of Bruneck (125 men and 125 women from each of the fifth through eighth decades of age, all White). In the present study, citrate plasma samples from the 2000 (n=668) follow-up were analysed. These samples were drawn after an overnight fast and 12 hours of abstinence from smoking. During the 2000 follow-up, citrate plasma was prepared by single centrifugation and aliquots were immediately stored at −80° C.

NMR

NMR-based lipoprotein profiling was conducted using the commercial Nightingale Health assay (Nightingale Health Ltd). This metabolic profiling platform enables the quantification of 14 lipoprotein subclasses defined as follows: extremely large-VLDL (>75 nm), five subclasses of VLDL (average particle diameter of 64.0 nm, 53.6 nm, 44.5 nm, 36.8 nm and 31.3 nm), intermediate density lipoprotein (IDL) (28.6 nm), three LDL subclasses (25.5 nm, 23.0 nm and 18.7 nm) and lastly four HDL subclasses (14.3 nm, 12.1 nm, 10.9 nm and 8.7 nm). The particle number of each lipoprotein subclass is quantified alongside lipid content including; phospholipids, cholesterol, free cholesterol, cholesterol esters and triglycerides. This NMR-based platform has been used previously in multiple epidemiological studies, where detailed technological information can be found(3-7).

Enzyme-Linked Immunosorbent Assay

Human PCSK9 concentrations were measured using the DuoSet ELISA Development kit (DY3888, R&D Systems) and the corresponding DuoSet Ancillary Reagent Kit 2 (R&D Systems) according to the manufacturer's instructions. Absorbance at 450 nm was measured on a plate reader (Tecan Infinite 200 Pro) using 570 nm as a reference wavelength. Concentrations were calculated using a 4-parameter logistic (4-PL) fit. Plasma was diluted 1:100, using 1 ul of plasma per sample, whereas 10 ug of HDL protein was diluted in 100 ul reagent diluent for this assay.

Lipoprotein-associated PCSK9 was measured using an in-house sandwich ELISA as previously described(8). Briefly, microtiter 96-well plates were coated overnight at 4° C. with alirocumab (5 mg/mL at 40 mL/well). Excess material was washed off and the plates blocked with 1% tris-buffered saline/bovine serum albumin for 45 minutes. EDTA plasma was added at 1:50 dilution (40 mL/well) for 75 minutes to allow alirocumab to bind PCSK9. This dilution of plasma provided conditions, whereby a saturating and equal amount of PCSK9 was captured in each well. To detect apoB-100, Lp(a), or apoAI bound to PCSK9 (PCSK9-apoB, PCSK9-Lp(a), and PCSK9-apoAI, respectively) biotinylated goat antihuman apoB-100 antibody (Academy Biomedical Co, Houston, Tex.) at 1 mg/mL, biotinylated murine monoclonal antibody LPA4 at 1 mg/mL or biotinylated goat anti-human APOA1 at 0.8 ng/mL, respectively, were added. Alkaline phosphatase-conjugated to NeutrAvidin (Thermo Scientific, Waltham, Mass.) was added for 60 minutes. Lumi-Phos 530 (Lumigen, Inc, Southfield, Mich.) (25 mL/well) was added for 75 minutes and luminescence read on a Dynex luminometer (Chantilly Technologies, Chantilly, Va.). The results are reported as RLU in 100 ms after subtraction of background RLU (tris-buffered saline/bovine serum albumin blank).

APOB Depletion (Liposep)

APOB containing lipoproteins were depleted from plasma using the Liposep APOB-specific immunoprecipitation reagent according to the manufacturer's instructions (Sun Diagnostics, New Gloucester, Me., USA). Plasma and immunoprecipitation reagent were mixed at a 1:1 ratio and incubated at room temperature for 10 minutes, with occasional vortex mixing. Samples were then centrifuged at 10,000×g for 10 minutes and the APOB depleted supernatant was taken, without disturbing the pellet, with aliquots being immediately stored at −80° C.

HDL-Immunodepletion

HDL was immuno-depleted from plasma using human HDL-specific IgY affinity columns according to manufacturer's instructions (Genway Biotech, San Diego, Calif., USA). Briefly, 40 ul of plasma was diluted 10-fold in 360 ul TBS buffer (10 mM Tris, 150 mM NaCl, pH 7.4). Diluted plasma was then added to TBS equilibrated antibody beads and incubated at room temperature with end over end rotation for 15 minutes. Flow through, HDL-depleted plasma, was then collected through centrifugation at 500×g. The removal of non-specifically bound proteins from the antibody beads was achieved using 500 ul of wash buffer (TBS, 0.05% Tween-20) a total of 3 times. HDL was then stripped from the antibody beads by the addition of 500 ul stripping buffer (0.1M Glycine, pH 2.5), twice. The antibody columns were then regenerated using a series of stripping buffer wash steps, followed by the addition of neutralisation buffer (100 mM Tris-HCl, pH 8.0) and lastly the resuspension in 500 ul TBS containing 0.02% sodium azide for storage. Isolated HDL samples were further concentrated, due to the large isolation volume and stored at −80° C. until further processing.

MRM-based Proteomics

Targeted quantitation of plasma proteins was conducted using the commercially available Plasma Dive kit (Biognosys) (9). Plasma samples were processed according to the manufacturer's instructions. Briefly, 10 ul of plasma was denatured and reduced in 90 ul of denature buffer and alkylated by the addition of 16 ul alkylation solution. 3 ul of alkylated protein (approximately 20 ug) were spiked with authentic heavy peptide standards (the peptide standard for apoB-100 did not overlap with the proximal portion of apoB that would include both apoB-48 and apoB-100). An in-solution tryptic digestion (Pierce Porcine Tryspin, enzyme:protein=1:50, Thermo Fisher Scientific) was performed overnight at 37° C. with shaking. Digestion was stopped by the addition of 10 ul, 10% TFA. After solid-phase extraction with C18 cartridges (Bravo AssayMAP, Agilent Technologies), the eluted peptides were dried using a SpeedVac (Thermo Fisher Scientific, Woburn, Mass.) and resuspended in 40 ul of liquid chromatography solution.

The samples were analysed on an Agilent 1290 Infinity II liquid chromatography system (Agilent Technologies, Santa Clara, Calif.) interfaced to an Agilent 6495 Triple Quadrupole MS (Agilent Technologies). Both instruments were controlled by MassHunter Workstation software (version B.08.00). The samples (10 ul) were directly injected onto a 25-cm column (AdvanceBio Peptide Mapping, C18, 2.1 mm×250 mm, 2.7 um, 120 Å, Agilent Technologies) and separated over a 23-minute gradient at 350 ul/min. Data files were analysed using SpectroDive 8 (Biognosys). Every peak integration was manually checked. Q-value <0.01 (FDR <1%) was used. The absolute concentration was calculated using Light/Heavy peptide signal intensity and known heavy peptide concentration.

Immunoblotting

Laemmli sample buffer (4×) (62.5 mM Tris-HCL, pH 6.8, 10% glycerol, 1% SDS, 0.005% bromophenol blue and 10% 2-mercaptoethanol) or without 2-mercaptoethanol was mixed with protein samples and boiled at 95° C. for 10 minutes. Protein samples were separated using 4-12% bis-tris gradient gels (Thermo scientific) in MOPS SDS running buffer (Thermo Scientific) at 130V for 90 minutes. Gels were either stained for total protein using SimplyBlue Safe Stain (Thermo Fisher) or proteins were transferred onto nitrocellulose membranes in ice-cold transfer buffer (25 mM tris-base pH 8.3; 192 mM glycine; 20% methanol) at 350 mA for 2 hours. Ponceau S red staining was used to determine efficient transfer and equal loading before membranes were blocked in 5% fat-free milk powder in phosphate-buffered saline (PBS) containing 0.1% Tween-20 (PBST)(Sigma). Membranes were incubated in primary antibodies (Table 1) made to appropriate concentrations in 5% BSA in PBST overnight at 4° C. The membranes were then incubated in the appropriate light-chain specific peroxidase-conjugated secondary antibody (Table I) in 5% milk/PBST. Membranes were then washed for three times in PBST for 15 minutes. Western blots were developed using enhanced chemiluminescence (ECL) (GE Healthcare) on photographic films (GE Healthcare). Densitometry analysis was done using the ImageJ analysis software.

Patient Information HDL CVD Cohort

From 2006 to 2007, blood samples were prospectively taken from consecutive patients ≥18 years of age presenting either with acute myocardial infarction (MI, ST-segment elevation myocardial infarction or non-ST-segment elevation or with stable coronary artery disease (sCAD) at the Clinic of Cardiology, West German Heart Center, University Hospital Essen (patients with MI and with sCAD) and the Alfried Krupp Hospital Essen (patients with MI).

Blood Collection and Plasma Preparation

In the MI group, blood sampling was performed during percutaneous coronary artery intervention for the treatment of the myocardial infarction as soon as the patient was clinically stabilized via the inserted arterial sheath or via an inserted venous catheter. In the sCAD group, study samples were collected during a routine blood sampling from peripheral veins. If a percutaneous coronary angiography had been performed in this study group for disease evaluation, the blood collection was undertaken on the day following the angiography to rule out an acute phase reaction upon vascular manipulation. In all groups, 30 ml of blood was drawn into vacuum tubes containing 1.6 mg EDTA/mL (4.298 mM EDTA/L). Immediately after blood drawing, the vacuum tubes were placed on ice and stored at 4° C. until further processing. Plasma was generated by centrifugation (3000 rpm, 30 minutes, 4° C.), immediately recovered and frozen at −80° C.

Ultracentrifugation-Based Isolation of High-Density Lipoprotein

All experimental procedures were performed by an investigator blinded to patients' data. High-density lipoproteins were isolated by sequential density gradient ultracentrifugation according to their density (1.069-1.21 g/mL), following an established protocol(10,11). Protein concentration was determined in each sample by Bradford assay (Bio-Rad, USA).

Postprandial Study Information

Postprandial samples were analysed from a double-blinded, 3-armed, randomised controlled trial (trial registration; clinicaltrials.gov NCT03191513; approved by King's College London Research Ethics Committee (HR-16/17-4397)) in healthy adults (n=20; 10 men, 10 women) aged 58 (SD 6.4) years. Samples were selected following consumption of the control test meal only containing 50 g rapeseed oil (61% 18:1n-9cis; 19% 18:2n-6cis) fed in the form of a muffin and a milkshake (to deliver 897 kcal, 50 g fat, 18 g protein, 88 g carbohydrate), following an overnight fast, a 50 g fat load has been shown to be the optimum quantity to discriminate between individual postprandial responses. Venous blood samples were collected at hourly intervals 0-8 h postprandially for analysis of plasma. Triacylglycerol (TAG) concentrations were measured on a Siemens ADVIA 1800 using the ADVIA chemistry TG method based on the Fossati three-step enzymatic reaction with a Trinder endpoint. A second, postprandial validation cohort was assessed (n=20, 8 time points), adhering to the same study design and test meal outlined above and has been previously published.¹⁰ Ethical approval for the study (ISRCTN20774126) was obtained from the relevant research ethics committees in the United Kingdom (NREC 08/H1101/122) and the Netherlands (MEC 09-3-009), and written informed consent was given by participants.

In-Solution Protein Digestion

HDL and Plasma samples were denatured by the addition of a final concentration of 6M urea and 2M thiourea and reduced by the addition of a final concentration of 10 mM DTT followed by incubation at 37° C. for 1 hour, 240 rpm. The samples were then cooled down to room temperature before being alkylated by the addition of a final concentration of 50 mM iodoacetamide followed by incubation in the dark for 30 minutes. Pre-chilled (−20° C.) acetone (10× volume) was used to precipitate the samples overnight at −20° C. Samples were centrifuged at 14000×g for 40 minutes at 4° C. and the supernatant subsequently discarded. Protein pellets were dried using a speed vac (Thermo Scientific, Savant SPD131DDA), resuspended in 0.1M TEAB buffer, pH 8.0, containing 0.02% ProteaseMax surfactant and mass spectrometry grade Trypsin/Lys-C (Promega Cooperation) (1:25 enzyme: protein) and digested overnight at 37° C., 240 rpm. Digestion was stopped by acidification with trifluoroacetic acid (TFA). Peptide samples were then purified by solid-phase extraction with C18 cartridges (Bravo AssayMAP, Agilent Technologies).

LC-MS/MS Analysis

The dried peptide samples for label free were reconstituted with 0.05% TFA in 2% ACN and separated by a nanoflow LC system (Dionex UltiMate 3000 RSLC nano). Samples were injected onto a nano-trap column (Acclaim® PepMap100 C18 Trap, 5 mm×300 um, 5 um, 100 Å), at a flow rate of 25 uL/min for 3 minutes, using 0.1% FA in H₂O. The following nano-LC gradient was then run at 0.25 uL/min to separate the peptides: 0-10 min, 4-10% B; 10-75 min, 10-30% B; 75-80 min, 30-40% B; 80-85 min, 40-99% B; 85-89.8 min, 99% B; 89.8-90 min, 99-4% B; 90-120 min, 4% B; where A=0.1% FA in H₂O, and B=80% ACN, 0.1% FA in H₂O. The nano column (EASY-Spray PepMap® RSLC C18, 2 pm 100 Å, 75 um×50 cm), set at 40° C. was connected to an EASY-Spray ion source (Thermo Scientific). Spectra were collected from an Orbitrap mass analyser (Orbitrap Fusion™ Lumos Tribrid, Thermo Scientific) using full MS mode (resolution of 120,000 at 400 m/z) over the mass-to-charge (m/z) range 375-1500. Data-dependent MS2 scan was performed using Quadrupole isolation in Top Speed mode using CID activation and ion trap detection in each full MS scan with dynamic exclusion enabled.

MS Database Search and Analysis

Thermo Scientific Proteome Discoverer software (version 2.2.0.388) was used to search raw data files against the human database, (UniProtKB/Swiss-Prot version 2018_02, 20,400 protein entries) using Mascot (version 2.6.0, Matrix Science). The mass tolerance was set at 10 ppm for precursor ions and 0.8 Da for fragment ions. Trypsin was used as the digestion enzyme with up to two missed cleavages being allowed.

Carbamidomethylation of cysteines and oxidation of methionine residues were chosen as fixed and variable modifications, respectively. MS/MS-based peptide and protein identifications were validated with the following filters, a peptide probability of greater than 95.0% (as specified by the Peptide Prophet algorithm), a protein probability of greater than 99.0%, and at least two unique peptides per protein. Data was normalized to the total peptide amount to take into account variation in abundances between samples.

Biocrates Lipidomics

Lipidomics analysis was conducted using Biocrates AbsoluteIDQ p400 (Biocrates Life Sciences AG, Innsbruck, Austria) kits according to manufacturer's instructions. 10 ul of internal standard (ISTD) was added to each well of the kit plate, followed by 10 ul of sample, QC or blank mixture into their respective wells. The plate was then dried using a Positive Pressure-96 Processor (Waters) for 30 minutes before the addition of 50 ul phenylidothiocyanate (PITC) derivatization solution (5% PITC, 31.7% ethanol, 31.7% pyridine, in H₂O) and was allowed to incubate at room temperature for 25 minutes. The plate was again dried using a pressure manifold and 300 ul of extraction buffer (5 mM ammonium acetate in methanol) was added to each well and incubated at room temperature, 450 rpm for 30 minutes. Lipid extracts were then collected by centrifugation, 500× g for 2 minutes. Extracts were then diluted in supplied FIA solvent and stored for no longer than overnight at 4° C. before analysis. Plasma and HDL lipid extracts were run by flow injection analysis (FIA), utilising the high resolution, accurate mass of a Q Exactive-Orbitrap MS coupled to a Vanquish Flex UHPLC system (Thermo Fisher), according to the manufacturer's specifications. Raw data was processed using the supplied MetIDQ software. Only Lipids that had a concentration greater than that of the limit of quantification were taken forward for analysis.

HepG2 Cell Culture

The human liver hepatocellular carcinoma cell line, HepG2 (ECACC 85011430), was used as in in vitro model of cellular cholesterol metabolism. Cells were cultured in Dulbecco's Modified Eagle's Medium (DMEM, Thermo Fisher Scientific) supplemented with 10% heat-inactivated foetal bovine serum (FBS), 2 mM L-glutamine and 1% penicillin/streptomycin (100 U/mL penicillin and 100 pg/mL streptomycin), at 37° C. in a humidified atmosphere of 95% air/5% CO₂.

HepG2 Cell Treatments and Protein Isolation

For PCSK9 studies cells were seeded in 6-well plates at a density of 3×10⁵ per well, and the next day media was changed to DMEM containing 10% lipoprotein deficient serum (LPDS, Merck). 24 h later media was changed and supplemented with stated concentrations of PCSK9 (ACRO Biosystems, PC9-H5223), reconstituted HDL (rHDL, Genway), ultracentrifuge-isolated HDL (ucHDL, Merck), after a prior pre-incubation at 37° C. for 1 h to promote PCSK9-HDL interaction, and Actinomycin D (Sigma, A9415) for 6 h. Cellular proteins were isolated by the following; cells were washed twice in ice cold PBS to eliminate secreted protein contamination before the addition of cell lysis buffer (25 mM Tris-HCL, 110 mM NaCl, 2 mM EGTA, 5 mM EDTA, 1% Triton and 0.5% SDS) supplemented with protease inhibitor cocktail (Roche), at pH 7.4. Cells were detached through scraping in cell lysis buffer and full lysis achieved by sonication and lysates were incubated on ice for 30 minutes. Cellular debris was then pelleted by centrifugation, 10,000×g, for 10 minutes at 4° C. Protein concentration was measured using the BCA protein assay kit (Thermo Fisher).

Cell Surface Protein Isolation

Cell surface proteins were isolated using the Pierce membrane protein isolation kit (Thermo Fisher) according to the manufacturer's instructions. Cells were washed twice with ice-cold PBS before incubation with Sulfo-NHS-SS-Biotin dissolved in PBS (0.25 mg/mL) on an orbital shaker for 30 minutes at 4° C. Membrane protein labelling was stopped using provided quenching solution and cells were scraped and centrifuged at 500×g for 1 minute and resulting pellets were washed twice with ice-cold PBS. Cells were lysed in lysis buffer supplemented with protease inhibitor (Complete mini-EDTA free Protease inhibitor cocktail, Roche) and proteins were solubilised through sonication; clarified lysates were then incubated with NeutrAvidin agarose for 60 minutes with end-over-end rocking. Membrane proteins were eluted from the NeutrAvidin beads through the incubation with cell lysis buffer containing 50 mM DTT.

Statistics

Proteomic and Lipidomic datasets were initially filtered to keep only molecules with less than 50% missing values. The remaining missing values were imputed using KNN-Impute method with k equal to the minimum value of 10 and the minimum samples assigned to each of the examined phenotypes (12). The relative quantities of the quantified molecules were further scaled using log 10 transformation.

All statistical comparisons have been conducted using non-parametric tests. Mann-Whitney U test was used for comparisons between two phenotypes and Kruskal Wallis test for comparisons between more than two phenotypes(13,14). P-values were adjusted using Benjamini Hochberg adjustment for multiple testing keeping proteins with false discovery rate threshold of 5%(15).

Pearson correlation, hierarchical cluster analysis and visualisation was conducted in the open-source software Perseus(16). All other data visualisations were created in Graphpad Prism (Version, 7.00, GraphPad Software, La Jolla Calif. USA). All reactions were carried out in 96-well plate format when possible, and liquid handling was performed using a Bravo AssayMAP robot (Agilent Technologies, Santa Clara, Calif., USA).

### REFERENCES FOR METHODS ONLY

1. Kaudewitz D, Skroblin P, Bender L H et al. Association of MicroRNAs and YRNAs With Platelet Function. Circulation research 2016; 118:420-432.
2. Langley S R, Willeit K, Didangelos A et al. Extracellular matrix proteomics identifies molecular signature of symptomatic carotid plaques. Journal of Clinical Investigation 2017; 127:1546-1560.
3. Soininen P, Kangas A J, Wurtz P et al. High-throughput serum NMR metabonomics for cost-effective holistic studies on systemic metabolism. The Analyst 2009; 134:1781-5.
4. Wurtz P, Tiainen M, Makinen V P et al. Circulating metabolite predictors of glycemia in middle-aged men and women. Diabetes care 2012; 35:1749-56.
5. Wurtz P, Wang Q, Kangas A J et al. Metabolic signatures of adiposity in young adults: Mendelian randomization analysis and effects of weight change. PLoS medicine 2014; 11:e1001765.
6. Soininen P, Kangas A J, Wurtz P, Suna T, Ala-Korpela M. Quantitative serum nuclear magnetic resonance metabolomics in cardiovascular epidemiology and genetics. Circulation Cardiovascular genetics 2015; 8:192-206.
7. Wurtz P, Kangas A J, Soininen P, Lawlor D A, Davey Smith G, Ala-Korpela M.

Quantitative Serum Nuclear Magnetic Resonance Metabolomics in Large-Scale Epidemiology: A Primer on—Omic Technologies. American journal of epidemiology 2017; 186:1084-1096.
8. Viney N J, Yeang C, Yang X, Xia S, Witztum J L, Tsimikas S. Relationship between “LDL-C”, estimated true LDL-C, apolipoprotein B-100, and PCSK9 levels following lipoprotein(a) lowering with an antisense oligonucleotide. Journal of clinical lipidology 2018; 12:702-710.
9. Yin X, Baig F, Haudebourg E et al. Plasma Proteomics for Epidemiology: Increasing Throughput With Standard-Flow Rates. Circulation Cardiovascular genetics 2017; 10.
10. Havel R J, Eder H A, Bragdon J H. The distribution and chemical composition of ultracentrifugally separated lipoproteins in human serum. Journal of Clinical Investigation 1955; 34:1345-53.
11. Kunitake S T, Kane J P. Factors affecting the integrity of high density lipoproteins in the ultracentrifuge. Journal of lipid research 1982; 23:936-40.
12. Zhang S. Nearest neighbor selection for iteratively kNN imputation. Journal of Systems and Software 2012; 85:2541-2552.
13. Hart A. Mann-Whitney test is not just a test of medians: differences in spread can be important. BMJ (Clinical research ed) 2001; 323:391-3.
14. Kruskal W H, Wallis W A. Use of Ranks in One-Criterion Variance Analysis. Journal of the American Statistical Association 1952; 47:583-621.
15. Benjamini Y, Hochberg Y. Controlling the False Discovery Rate: A Practical and Powerful Approach to Multiple Testing. Journal of the Royal Statistical Society Series B (Methodological) 1995; 57:289-300.
16. Tyanova S, Temu T, Sinitcyn P et al. The Perseus computational platform for comprehensive analysis of (prote)omics data. Nature Methods 2016; 13:731.

TABLE I

| Target | Host | | Company, Catalogue |
|---|---|---|---|
| Protein | Species | Application | Number |
| PCSK9 | Sheep | Immunoblotting (1:1000) | R&D Systems, AF3888 |
| APOA1 | Rabbit | Immunoblotting (1:1000) | Abcam, ab52945 |
| HRP-anti- | Donkey | Immunoblotting (1:1000) | R&D Systems, HAF016 |
| Sheep | | | |
| HRP-anti- | Mouse | Immunoblotting (1:5000) | Jackson Immuno |
| Rabbit | | | Research, 211032171 |

### REFERENCES FOR DESCRIPTION AND EXAMPLES 1-8

1. Roth G A, Johnson C, Abajobir A et al. Global, Regional, and National Burden of Cardiovascular Diseases for 10 Causes, 1990 to 2015. Journal of the American College of Cardiology 2017; 70:1-25.
2. Gordon T, Castelli W P, Hjortland M C, Kannel W B, Dawber T R. High density lipoprotein as a protective factor against coronary heart disease: The Framingham study. The American Journal of Medicine 1977; 62:707-714.
3. Boekholdt S M, Arsenault B J, Hovingh G K et al. Levels and Changes of HDL Cholesterol and Apolipoprotein A-1 in Relation to Risk of Cardiovascular Events Among Statin-Treated Patients. Circulation 2013; 128:1504-1512.
4. Toth P P, Barter P J, Rosenson R S et al. High-density lipoproteins: a consensus statement from the National Lipid Association. Journal of clinical lipidology 2013; 7:484-525.
5. Cannon C P, Shah S, Dansky H M et al. Safety of anacetrapib in patients with or at high risk for coronary heart disease. The New England journal of medicine 2010; 363:2406-15.
6. Schwartz G G, Olsson A G, Abt M et al. Effects of dalcetrapib in patients with a recent acute coronary syndrome. The New England journal of medicine 2012; 367:2089-99.
7. Boden W E, Probstfield J L, Anderson T et al. Niacin in patients with low HDL cholesterol levels receiving intensive statin therapy. The New England journal of medicine 2011; 365:2255-67.
8. HPS2-THRIVE randomized placebo-controlled trial in 25 673 high-risk patients of ER niacin/laropiprant trial design, pre-specified muscle and liver outcomes, and reasons for stopping study treatment. European heart journal 2013; 34:1279-91.
9. Barter P J, Caulfield M, Eriksson M et al. Effects of torcetrapib in patients at high risk for coronary events. The New England journal of medicine 2007; 357:2109-22.
10. Group THTRC. Effects of Anacetrapib in Patients with Atherosclerotic Vascular Disease. New England Journal of Medicine 2017; 377:1217-1227.
11. Voight B F, Peloso G M, Orho-Melander M et al. Plasma HDL cholesterol and risk of myocardial infarction: a mendelian randomisation study. Lancet (London, England) 2012; 380:572-80.
12. Holmes M V, Asselbergs F W, Palmer T M et al. Mendelian randomization of blood lipids for coronary heart disease. European heart journal 2015; 36:539-50.
13. Pechlaner R, Tsimikas S, Yin X et al. Very-Low-Density Lipoprotein-Associated Apolipoproteins Predict Cardiovascular Events and Are Lowered by Inhibition of APOC-III. Journal of the American College of Cardiology 2017; 69:789-800.
14. Riwanto M, Rohrer L, Roschitzki B et al. Altered activation of endothelial anti- and proapoptotic pathways by high-density lipoprotein from patients with coronary artery disease: role of high-density lipoprotein-proteome remodeling. Circulation 2013; 127:891-904.
15. O'Reilly M, Dillon E, Guo W et al. High-Density Lipoprotein Proteomic Composition, and not Efflux Capacity, Reflects Differential Modulation of Reverse Cholesterol Transport by Saturated and Monounsaturated Fat Diets. Circulation 2016; 133:1838-50.
16. Besler C, Heinrich K, Rohrer L et al. Mechanisms underlying adverse effects of HDL on eNOS-activating pathways in patients with coronary artery disease. Journal of Clinical Investigation 2011; 121:2693-708.
17. Holmes M V, Millwood I Y, Kartsonaki C et al. Lipids, Lipoproteins, and Metabolites and Risk of Myocardial Infarction and Stroke. Journal of the American College of Cardiology 2018; 71:620-632.
18. Zhang D W, Lagace T A, Garuti R et al. Binding of proprotein convertase subtilisin/kexin type 9 to epidermal growth factor-like repeat A of low density lipoprotein receptor decreases receptor recycling and increases degradation. The Journal of biological chemistry 2007; 282:18602-12.
19. Sabatine M S, Giugliano R P, Keech A C et al. Evolocumab and Clinical Outcomes in Patients with Cardiovascular Disease. The New England journal of medicine 2017; 376:1713-1722.
20. Schwartz G G, Steg P G, Szarek M et al. Alirocumab and Cardiovascular Outcomes after Acute Coronary Syndrome. The New England journal of medicine 2018; 379:2097-2107.
21. Kosenko T, Golder M, Leblond G, Weng W, Lagace T A. Low Density Lipoprotein Binds to Proprotein Convertase Subtilisin/Kexin Type-9 (PCSK9) in Human Plasma and Inhibits PCSK9-mediated Low Density Lipoprotein Receptor Degradation. The Journal of biological chemistry 2013; 288:8279-88.
22. Tavori H, Christian D, Minnier J et al. PCSK9 Association With Lipoprotein(a). Circulation research 2016; 119:29-35.
23. Wurtz P, Kangas A J, Soininen P, Lawlor D A, Davey Smith G, Ala-Korpela M. Quantitative Serum Nuclear Magnetic Resonance Metabolomics in Large-Scale Epidemiology: A Primer on—Omic Technologies. American journal of epidemiology 2017; 186:1084-1096.
24. Soininen P, Kangas A J, Wurtz P, Suna T, Ala-Korpela M. Quantitative serum nuclear magnetic resonance metabolomics in cardiovascular epidemiology and genetics. Circulation Cardiovascular genetics 2015; 8:192-206.
25. Lakoski S G, Lagace T A, Cohen J C, Horton J D, Hobbs H H. Genetic and metabolic determinants of plasma PCSK9 levels. The Journal of clinical endocrinology and metabolism 2009; 94:2537-43.
26. Mayne J, Dewpura T, Raymond A et al. Plasma PCSK9 levels are significantly modified by statins and fibrates in humans. Lipids in health and disease 2008; 7:22.
27. Masson D, Jiang X C, Lagrost L, Tall A R. The role of plasma lipid transfer proteins in lipoprotein metabolism and atherogenesis. Journal of lipid research 2009; 50 Suppl:S201-6.
28. Campos E, McConathy W J. Distribution of lipids and apolipoproteins in human plasma by vertical spin ultracentrifugation. Arch Biochem Biophys 1986; 249:455-63.
29. Xu R X, Li S, Zhang Y et al. Relation of plasma PCSK9 levels to lipoprotein subfractions in patients with stable coronary artery disease. Lipids in health and disease 2014; 13:188.
30. Koren M J, Kereiakes D, Pourfarzib R et al. Effect of PCSK9 Inhibition by Alirocumab on Lipoprotein Particle Concentrations Determined by Nuclear Magnetic Resonance Spectroscopy. Journal of the American Heart Association 2015; 4.
31. Sliz E, Kettunen J, Holmes M V et al. Metabolomic Consequences of Genetic Inhibition of PCSK9 Compared With Statin Treatment. Circulation 2018; 138:2499-2512.
32. Romagnuolo R, Scipione C A, Boffa M B, Marcovina S M, Seidah N G, Koschinsky M L. Lipoprotein(a) catabolism is regulated by proprotein convertase subtilisin/kexin type 9 through the low density lipoprotein receptor. The Journal of biological chemistry 2015; 290:11649-62.
33. Munroe W H, Phillips M L, Schumaker V N. Excessive centrifugal fields damage high density lipoprotein. Journal of lipid research 2015; 56:1172-81.
34. Tavori H, Giunzioni I, Linton M F, Fazio S. Loss of plasma proprotein convertase subtilisin/kexin 9 (PCSK9) after lipoprotein apheresis. Circulation research 2013; 113:1290-5.
35. Julius U, Milton M, Stoellner D et al. Effects of lipoprotein apheresis on PCSK9 levels. Atherosclerosis Supplements 2015; 18:180-6.
36. Lagace T A, Curtis D E, Garuti R et al. Secreted PCSK9 decreases the number of LDL receptors in hepatocytes and in livers of parabiotic mice. Journal of Clinical Investigation 2006; 116:2995-3005.
37. Ray K K, Landmesser U, Leiter L A et al. Inclisiran in Patients at High Cardiovascular Risk with Elevated LDL Cholesterol. The New England journal of medicine 2017; 376:1430-1440.
38. Poirier S, Mayer G, Poupon V et al. Dissection of the Endogenous Cellular Pathways of PCSK9-induced Low Density Lipoprotein Receptor Degradation: EVIDENCE FOR AN INTRACELLULAR ROUTE. Journal of Biological Chemistry 2009; 284:28856-28864.
39. Silver D L, Wang N, Xiao X, Tall A R. High density lipoprotein (HDL) particle uptake mediated by scavenger receptor class B type 1 results in selective sorting of HDL cholesterol from protein and polarized cholesterol secretion. The Journal of biological chemistry 2001; 276:25287-93.
40. Rashid S, Tavori H, Brown P E et al. Proprotein convertase subtilisin kexin type 9 promotes intestinal overproduction of triglyceride-rich apolipoprotein B lipoproteins through both low-density lipoprotein receptor-dependent and -independent mechanisms. Circulation 2014; 130:431-41.
41. Sun H, Samarghandi A, Zhang N, Yao Z, Xiong M, Teng B B. Proprotein convertase subtilisin/kexin type 9 interacts with apolipoprotein B and prevents its intracellular degradation, irrespective of the low-density lipoprotein receptor. Arteriosclerosis, thrombosis, and vascular biology 2012; 32:1585-95.
42. Lambert G, Jamoux A L, Pineau T et al. Fasting induces hyperlipidemia in mice overexpressing proprotein convertase subtilisin kexin type 9: lack of modulation of very-low-density lipoprotein hepatic output by the low-density lipoprotein receptor. Endocrinology 2006; 147:4985-95.
43. Ooi T C, Krysa J A, Chaker S et al. The Effect of PCSK9 Loss-of-Function Variants on the Postprandial Lipid and ApoB-Lipoprotein Response. The Journal of clinical endocrinology and metabolism 2017; 102:3452-3460.
44. Cariou B, Langhi C, Le Bras M et al. Plasma PCSK9 concentrations during an oral fat load and after short term high-fat, high-fat high-protein and high-fructose diets. Nutrition & metabolism 2013; 10:4.
45. Wojczynski M K, Glasser S P, Oberman A et al. High-fat meal effect on LDL, HDL, and VLDL particle size and number in the Genetics of Lipid-Lowering Drugs and Diet Network (GOLDN): an interventional study. Lipids in health and disease 2011; 10:181.
46. Baass A, Dubuc G, Tremblay M et al. Plasma PCSK9 is associated with age, sex, and multiple metabolic markers in a population-based sample of children and adolescents. Clinical chemistry 2009; 55:1637-45.
47. Ly K, Essalmani R, Desjardins R, Seidah N G, Day R. An Unbiased Mass Spectrometry Approach Identifies Glypican-3 as an Interactor of Proprotein Convertase Subtilisin/Kexin Type 9 (PCSK9) and Low Density Lipoprotein Receptor (LDLR) in Hepatocellular Carcinoma Cells. Journal of Biological Chemistry 2016; 291:24676-24687.
48. Subramaniam S, Jurk K, Hobohm L et al. Distinct contributions of complement factors to platelet activation and fibrin formation in venous thrombus development Blood 2017; 129:2291-2302.
49. Bajic G, Degn S E, Thiel S, Andersen G R. Complement activation, regulation, and molecular basis for complement-related diseases. The EMBO journal 2015; 34:2735-57.
50. Walley K R, Francis G A, Opal S M, Stein E A, Russell J A, Boyd J H. The Central Role of Proprotein Convertase Subtilisin/Kexin Type 9 in Septic Pathogen Lipid Transport and Clearance. American journal of respiratory and critical care medicine 2015; 192:1275-86.
51. Walley K R, Thain K R, Russell J A et al. PCSK9 is a critical regulator of the innate immune response and septic shock outcome. Science translational medicine 2014; 6:258ra143.
52. Shapiro M D, Tavori H and Fazio S. PCSK9: From Basic Science Discoveries to Clinical Trials. Circulation research. 2018; 122:1420-1438.

1. A method of assessing unbound PCSK9 in a subject the method comprising:
(a) providing a blood sample from the subject who is optionally diagnosed with, or believed to be at risk of, CVD;
(b) specifically depleting at least HDL from the sample to remove HDL-bound PCSK9 from the sample;
(c) assessing the level of unbound PCSK9 from the depleted sample.

2. A method as claimed in claim 1 further comprising assessing either total PCSK9 or PCSK9 bound to the HDL from the sample.

3. A method of assessing PCSK9 activity in a subject the method comprising:
(a) providing a blood sample from the subject who is optionally diagnosed with, or believed to be at risk of, CVD;
(b) assessing the amount of PCSK9 bound to the HDL from the sample, optionally by specifically depleting HDL from the sample to remove HDL-bound PCSK9 from the sample;
(c) optionally assessing the amount of LDL-bound PCSK9 from the sample, optionally by specifically depleting ApoB and/or LDL from the sample;
(d) correlating the amount of PCSK9 bound to HDL, or the ratio of PCSK9 bound to HDL compared to bound to LDL, with the PCSK9 activity.

4. A method as claimed in claim 3 wherein the ratio of PCSK9 bound to HDL compared to LDL bound is correlated with the PCSK9 activity

5. A method as claimed in any one of the preceding claims wherein the blood sample is a serum sample or plasma sample.

6. A method as claimed in any one of the preceding claims wherein the method comprises specifically depleting ApoB and/or LDL from the sample to remove LDL-bound PCSK9 from the sample.

7. A method as claimed in any one of the preceding claims wherein PCSK9 in the subject is assessed postprandially, optionally following a standard meal preceded by a period of fasting.

8. A method as claimed in any claim 7 wherein the level of unbound and bound PCSK9 or PCSK9 activity is assessed over a period of time postprandially, which is optionally up to 3, 4, 5, 6, 7 or 8 hours.

9. A method as claimed in any claim 8 wherein the unbound and bound PCSK9 or PCSK9 activity over the period of time are subject to area under the curve analysis for the subject.

10. A method as claimed in any one of the preceding claims wherein the subject is individually assessed.

11. A method as claimed in any one of the preceding claims wherein the subject is part of a subject group who are optionally diagnosed with, or believed to be at risk of, CVD, all of whom are assessed.

12. A method as claimed in any claim 11 wherein the group are stratified according to the result of the level of unbound PCSK9 from the depleted sample, and optionally the PCSK9 bound to the HDL from the sample and/or PCSK9 activity.

13. A method as claimed in any one of the preceding claims wherein the level of unbound PCSK9 from the depleted sample or PCSK9 activity is compared to a control, reference or threshold level.

14. A method as claimed in claim 13 wherein the reference level for unbound PCSK9 is the PCSK9 bound to the HDL from the sample or total PCSK9 in the sample, wherein optionally the ratio of unbound: HDL bound or unbound: total PCSK9 is calculated.

15. A method as claimed in claim 13 wherein the reference level is a measure of central tendency of unbound PCSK9 or PCSK9 activity, which is optionally a mean level, observed in one or more populations, wherein the one or more populations are optionally selected from a responsive group of subjects who have responded positively to treatment with a compound which is a statin or an inhibitor or putative inhibitor of PCSK9 or a non-responsive group of subjects who have not responded positively to treatment with a compound which is a statin or an inhibitor or putative inhibitor of PCSK9.

16. A method as claimed in claim 13 wherein the reference level is based on past measurements of unbound PCSK9 or PCSK9 activity in the same subject.

17. A method as claimed in any one of claims 1 to 16 wherein the depletion in step (b) or (c) is performed by one or more of (i) column chromatography, which is optionally immunodepletion (ii) centrifugation, (iii) electrophoresis, or (iv) precipitation, which is optionally immunoprecipitation.

18. A method as claimed in claim 17 wherein the column chromatography is selected from affinity chromatography, size exclusion chromatography, which is optionally HPLC.

19. A method as claimed in any one of claims 1 to 18 wherein the assessing, optionally in in step (c), is performed using an immunoassay; an aptamer-based method; or mass spectrometry.

20. A method as claimed in any one of claims 1 to 19 wherein the level of unbound PCSK9 from the depleted sample is calculated by subtracting PCSK9 bound to the HDL from the sample from total PCSK9 in the sample.

21. A method of:
selecting a subject for treatment with a compound which is a statin or an inhibitor or putative inhibitor of PCSK9; or
classifying a subject according to their likelihood of responding to treatment with a compound which is a statin or an inhibitor or putative inhibitor of PCSK9; or
predicting the response of a subject to treatment with a compound which is a statin or an inhibitor or putative inhibitor of PCSK9; or
determining whether an anti-CVD effect is likely to be produced in a subject by treatment with a compound which is a statin or an inhibitor of PCSK9; or
estimating the level of in vivo binding of an antibody directed against PCSK9 in the subject
assessing the response of a subject who has previously been treated with a statin or an inhibitor or putative inhibitor of PCSK9;
the method comprising:
(i) performing a method of assessing unbound PCSK9 or PCSK9 activity in the subject according to any one of claims 1 to 20
(ii) using the result of the level of unbound PCSK9 from the depleted sample or PCSK9 activity, and optionally the PCSK9 bound to the HDL from the sample to respectively: select the subject; classify the subject; predict the response; determine whether an anti-CVD effect is likely to be produced; estimate the level of in vivo binding of an antibody directed against PCSK9 in the subject; assess the response.

22. A method as claimed in any one of claims 1 to 21 further comprising treating or further treating a subject selected in accordance with the level of unbound PCSK9 or PCSK9 activity from the depleted sample, and optionally the PCSK9 bound to the HDL from the sample, with a compound which is a statin or inhibitor or putative inhibitor of PCSK9.

23. A method for assessing the efficacy of a compound which is a statin or an inhibitor or putative inhibitor of PCSK9 which is putatively therapeutic for CVD, the method comprising the steps of:
(a) selecting a treatment group who have been diagnosed with, or believed to be at risk of, CVD and who have been classified as being likely to be responsive to treatment with such a compound according to a method of claim 21;
(b) treating members of the treatment group with the compound for a treatment timeframe;
(c) deriving physiological outcome measures for the treatment group;
(d) comparing the outcomes at (d) with a comparator arm of which is optionally a placebo or minimal efficacy comparator arm;
(e) using the comparison in (d) to derive an efficacy measure for the compound.

24. A method of treating CVD comprising administering a compound which is a statin or an inhibitor of PCSK9 to a subject that has been determined to be responsive to the compound based on the level of serum PCKS9 in the subject not bound to HDL or the proportion of the non-bound PCSK9 to the HDL-bound PCSK9 or to the total PCSK9 or the PCSK9 activity.

25. A method of treating CVD comprising administering a compound which is a statin or an inhibitor of PCSK9 to a subject, wherein the subject has previously been selected for such treatment according to the method of claim 21.

26. A method of treating CVD comprising administering a compound which is a statin or an inhibitor of PCSK9 to a subject, wherein the method comprises selecting the subject for such treatment according to a method of claim 21.

27. A compound which is a statin or an inhibitor or putative inhibitor of PCSK9 for use in a method of treating a subject diagnosed with, or believed to be at risk of, CVD,
wherein the subject that has been determined to be responsive to the compound based on the level of expression of PCKS9 in the subject not bound to HDL or the proportion of the non-bound PCSK9 to the HDL-bound PCSK9 or to the total PCSK9 or the PCSK9 activity.

28. A compound which is a statin or inhibitor or putative inhibitor of PCSK9 for use in a method of treating a subject diagnosed with, or believed to be at risk of, CVD,
wherein the subject has previously been selected for such treatment according to the method of claim 21.

29. A compound which is a statin or an inhibitor or putative inhibitor of PCSK9 for use in a method of treating a subject diagnosed with, or believed to be at risk of, CVD,
wherein the method comprises selecting the subject for such treatment according to a method of claim 21.

30. Use of a compound which is a statin or an inhibitor of PCSK9 in the preparation of a medicament for use in a method of treating a subject diagnosed with, or believed to be at risk of, CVD,
wherein the subject that has been determined to be responsive to the compound based on the level of expression of PCKS9 in the subject not bound to HDL or the proportion of the non-bound PCSK9 to the HDL-bound PCSK9 or to the total PCSK9 or the PCSK9 activity.

31. Use of a compound which is a statin or an inhibitor of PCSK9 in the preparation of a medicament for use in a method of treating a subject diagnosed with, or believed to be at risk of, CVD,
wherein the subject has previously been selected for such treatment according to the method of claim 21.

32. Use of a compound which is a statin or an inhibitor of PCSK9 in the preparation of a medicament for use in a method of treating a subject diagnosed with, or believed to be at risk of, CVD,
wherein the method comprises selecting the subject for such treatment according to a method of claim 21.

33. A method, compound for use, or use according to claim 15 or any one of claims 21 to 32 wherein the compound is a statin.

34. A method, compound for use, or use according to claim 15 or any one of claims 21 to 32 wherein the compound is an inhibitor of PCSK9.

35. A method, compound for use, or use according to claim 15 or any one of claims 21 to 32 wherein the compound binds directly to PCSK9, inhibiting its interaction with LDLR and/or intemalisation of LDLR and/or targeting of LDLR for lysosomal degradation.

36. A method, compound for use, or use according to claim 15 or any one of claims 21 to 32 wherein the compound is an antibody molecule.

37. A method, compound for use, or use according to claim 15 or any one of claims 21 to 32 wherein the compound is selected from Table T.

38. A method, compound for use, or use according to any one of claims 1 to 37 wherein said CVD comprises at least one of coronary atherosclerosis, dyslipidemia, type II dyslipidemia, hypercholesterolemia and myocardial infarction.

39. A kit for use in a method of any one of claims 1 to 26 which comprises:
(a) means for collecting serum, plasma or full blood from the subject; and/or
(b) means for specifically depleting at least HDL from the sample to remove bound PCSK9 from the sample; and/or
(c) means assessing the level of PCSK9 from the depleted plasma sample; and
(d) instructions for use in the method.

40. A kit as claimed in claim 39 which comprises means for specifically depleting ApoB and/or LDL from the sample.
